(19) United States
(12) Patent Application Publication
(10) Pub. No.: US 2026/0125635 A1
(43) Pub. Date: May 7, 2026

(54) NEW LACTIC ACID BACTERIA

(71) Applicant: INTERNATIONAL N&H DENMARK APS, KONGENS LYNGBY (DK)

(72) Inventors: Anais JEDRZEJOWSKI, Dangé-Saint-Romain (FR); Christophe FREMAUX, Dangé-Saint-Romain (FR); Sabine VAN DILLEN, Dangé-Saint-Romain (FR); Thomas DESFOUGERES, Dangé-Saint-Romain (FR); Max Charles JODEAU, Dangé-Saint-Romain (FR); Damien LUGAND, Dangé-Saint-Romain (FR)

(21) Appl. No.: 19/328,812

(22) Filed: Sep. 15, 2025

Related U.S. Application Data
(63) Continuation of application No. 17/437,255, now Pat. No. 12,441,977.

(30) Foreign Application Priority Data
Mar. 14, 2019 (EP) 19162856.9
May 16, 2019 (CN) 201910406044.7
Dec. 6, 2019 (EP) 19214119.0

(51) Int. Cl. C12N 1/20; A23C 9/123; A23L 13/74; C12N 15/74; C12P 1/04; G01N 33/50

(52) U.S. Cl. CPC C12N 1/20; A23C 9/1238; A23L 13/74; C12N 15/746; C12P 1/04; G01N 33/5038; A23V 2400/249

(57) ABSTRACT

The invention relates to a polynucleotide comprising a lacZ gene (lacZ^{FS}) encoding a β-galactosidase characterized by a particular profile regarding its efficiency of hydrolysis of lactose. The invention is also directed to a Streptococcus thermophilus strain comprising a lacZ^{FS} allele and bacterial composition thereof, and their use to obtain fermented milk not undergoing post-acidification.

### CROSS-REFERENCE TO RELATED APPLICATIONS

The present application is a Continuation of U.S. application Ser. No. 17/437,255, filed Sep. 8, 2021, which is a U.S. national stage application under 35 U.S.C. § 371 of International Application No. PCT/EP2020/056766, filed on Mar. 13, 2020, entitled “NEW LACTIC ACID BACTERIA,” and claims priority from EP Application No. 19162856.9, filed Mar. 14, 2019, CN Application No. 201910406044.7, filed May 16, 2019, and EP Application No. 19214119.0, filed Dec. 6, 2019, the contents of which are incorporated by reference in their entirety.

### FIELD OF THE INVENTION

The invention relates to a polynucleotide comprising a lacZ gene (lacZ^{FS}) encoding a β-galactosidase characterized by a particular profile regarding its efficiency of hydrolysis of lactose. The invention is also directed to a Streptococcus thermophilus strain comprising a lacZ^{FS} allele and bacterial composition thereof, and their use to obtain fermented milk not undergoing post-acidification.

### INCORPORATION BY REFERENCE OF THE SEQUENCE LISTING

The present application is being filed with a Sequence Listing in electronic format. The Sequence Listing is provided as a file entitled 20250912_NB41527USPCN_SequenceListing.xml, created on Sep. 12, 2025, which is 76,618 bytes in size. The information in the electronic format of the Sequence Listing is incorporated by reference in its entirety.

### BACKGROUND TO THE INVENTION

The food industry uses bacteria in order to improve the taste and the texture of food or feed products. In the case of the dairy industry, lactic acid bacteria are commonly used in order to, for example, bring about the acidification of milk (by fermentation of lactose) and to texturize the product into which they are incorporated. For example, the lactic acid bacteria of the species Streptococcus thermophilus (S. thermophilus) are used extensively, alone or in combination with other bacteria, in the manufacture of fresh fermented dairy products, such as cheese or yoghurt.

One of the limitations of the use of lactic acid bacteria in dairy technology is post-acidification, i.e. the production of lactic acid by the lactic acid bacteria after the target pH (the one required by the technology) has been obtained. Thus, to avoid this post-acidification phenomenon, dairy product manufacturers are required to rapidly cool the fermented product right after the target pH is obtained. Thus, dairy product manufacturers lack flexibility in the manufacturing process, while having the possibility of keeping the fermented product at the fermentation temperature for some time would be an advantage. Moreover, the cooling step is energy-consuming, such that bypassing the cooling step, would be both an economical and environmental advantage.

WO90/05459 describes Lactobacillus bulgaricus mutant strains, selected based on their color phenotype on X-gal-containing medium. The application reports the identification of temperature conditional L. bulgaricus mutants (blue at 37° C., but white at 4° C.) and pH sensitive L. bulgaricus mutants (blue at pH 7 but white at pH 4.5 or 5). However, WO90/05459 is silent about any mutation in the lacZ gene. Moreover, WO90/05459 describes mutants characterized by enzyme which has an activity of at least 90% the activity of a wild type enzyme in production conditions (processing temperature or processing pH), while having an activity reduced of at least 20% as compared to the activity of a wild type enzyme in storage conditions. However, the teaching of WO90/05459 is insufficient regarding any enzyme activity and in particular regarding the beta-galactosidase activity; indeed, as shown in examples 4 and 5 of the present application, there is neither admitted reference beta-galactosidase activity in strains, at pH 4.5 or pH 6. Therefore, the characterization of the mutants described in WO90/015459 is not possible without any reference value or reference strain.

WO2010/139765 describes a method to manufacture a fermented dairy product using a weakly post-acidifying culture based on specific Lactobacillus bulgaricus strains. Because the culture is characterized by a weak production of lactic acid at fermentation temperature, the pH is substantially steady and the cooling step can be avoided. However, WO2010/139765 does not characterize the exemplified Lactobacillus bulgaricus strains.

WO2015/193459 proposes other solutions to overcome the post-acidification issue: controlling the concentration of lactose in the milk before fermentation for example by adding lactase, providing lactic acid bacteria which are not able to hydrolyze lactose (lactose-deficient lactic acid bacteria). These solutions are however not satisfactory for dairy product manufacturers, since they require either the addition of exogenous enzyme (such as lactase) in the milk before fermentation rendering the manufacturing process more complex and more expensive, or the addition of a carbohydrate into the milk (such as sucrose) what is not in agreement with the growing demand for healthier products with no additives.

Therefore, there is a need for providing means to dairy product manufacturers, for producing fermented products based on lactic acid bacteria, with both satisfactory results and high flexibility in the manufacturing process.

### DESCRIPTION OF THE DRAWINGS

FIG. 1 are graphs representing (A) the acidification profile in milk (pH over time) of DGCC7984 strain and its two subclones DGCC12455 and DGCC12456, and (B) the evolution of the speed of acidification over time (mUpH/min over time) of strain DGCC12456

FIG. 2 are graphs representing (A) the acidification profile in milk (pH over time) and (B) the evolution of the speed of acidification over time (mUpH/min over time), of strain DGCC715

FIG. 3 are graphs representing (A) the acidification profile in milk (pH overtime) and (B) the evolution of the speed of acidification over time (mUpH/min overtime), of strain 715^{R354C}

FIG. 4 are graphs representing (A) the acidification profile in milk (pH overtime) and (B) the evolution of the speed of acidification over time (mUpH/min over time), of strain DGCC11231

FIG. 5 are graphs representing (A) the acidification profile in milk (pH overtime) and (B) the evolution of the speed of acidification overtime (mUpH/min overtime), of strain 11231^{R354C}

FIG. 6 is a graph representing the beta-galactosidase activity at pH6 and pH 4.5 of four S. thermophilus strains

FIG. 7 is a graph representing the beta-galactosidase activity at pH6 and pH 4.5 of strain DGCC715, strain 715^{R354C}, strain DGCC11231, strain 11231^{R354C} and strain DGCC12456

FIG. 8 is a graph representing the ratio LacS over LacZ at pH6 and pH 4.5 of strain DGCC715, strain 715^{R354C}, strain DGCC11231, strain 11231^{R354C} and strain DGCC12456

FIG. 9 is a graph representing the difference of efficiency of hydrolysis of lactose between pH6 and pH 4.5 (ΔEH) of strain DGCC715, strain 715^{R354C}, strain DGCC11231, strain 11231^{R354C} and strain DGCC12456.

FIG. 10 is a graph representing (A) the viscosity measured on day 14 and (B) the evolution of pH over time, for a stirred yoghurt manufactured with strain DGCC12456 and packed at a temperature of 20° C. or 35° C. (storage at 10° C.).

FIG. 11 is a graph representing the evolution of pH over time of a yoghurt manufactured with strain DGCC12456 (plain line) and with a reference culture (dashed line) (stored at 10° C.).

### SUMMARY OF THE INVENTION

In one aspect, the invention is directed to a polynucleotide encoding a β-galactosidase^{FS}, which, when inserted in lieu of the allele of the lacZ gene of strain DGCC715 (deposited at the DSMZ on Feb. 12, 2019 under the accession number DSM33036), leads to a DGCC715-derivative characterized by a ratio LacS_{pH4.5} over LacZ_{pH4.5} which is more than 8, wherein LacS_{pH4.5} represents the activity of lactose importation of the LacS permease calculated by assay A at pH 4.5, and LacZ_{pH4.5} represents the activity of lactose hydrolysis of the beta-galactosidase calculated by assay B at pH 4.5. Thus, the invention is directed to a polynucleotide encoding a β-galactosidase^{FS}, which is defined as a lacZ allele which increases the ratio of the activity of lactose importation of the LacS permease calculated by assay A at pH 4.5 over the activity of lactose hydrolysis of the beta-galactosidase calculated by assay B at pH 4.5 (ratio LacS_{pH4.5} over LacZ_{pH4.5}) above 8 in a DGCC715 derivative, said DGCC715 derivative being a strain DGCC715 (deposited at the DSMZ on Feb. 12, 2019 under the accession number DSM33036), into which its lacZ gene was replaced by said polynucleotide encoding a β-galactosidase^{FS}.

In one aspect, the invention is directed to a polynucleotide comprising a part of at least 100 nucleotides of the polynucleotide encoding a β-galactosidase^{FS}, wherein said nucleotide part encompasses the codon corresponding to the residue 354 of said β-galactosidase^{FS}. In one aspect, the invention is directed to a vector comprising a polynucleotide of the invention.

In one aspect, the invention is directed to a Streptococcus thermophilus strain comprising an allele of the lacZ gene which is a lacZ^{FS} allele encoding a β-galactosidase^{FS} according to the invention.

In one aspect, the invention is directed to a bacterial composition comprising the Streptococcus thermophilus strain of the invention.

In one aspect, the invention is directed to a food or feed product comprising the Streptococcus thermophilus strain of the invention or the bacterial composition of the invention.

In one aspect, the invention is directed to a method for manufacturing a fermented product, comprising: a) inoculating a substrate with the Streptococcus thermophilus strain of the invention or the bacterial composition of the invention; and b) fermenting the inoculated substrate obtained from step a) to obtain a fermented product, preferably a fermented dairy product.

In one aspect, the invention is directed to the use of the Streptococcus thermophilus strain of the invention or the bacterial composition of the invention, to manufacture a food or feed product, preferably a fermented food product, more preferably a fermented dairy product.

In one aspect, the invention is directed to the use of a polynucleotide or vector of the invention, to obtain a Streptococcus thermophilus strain with a full STOP phenotype when used to ferment milk by assay C.

In one aspect, the invention is directed to a method to prepare a Streptococcus thermophilus strain with a full STOP phenotype, comprising: a) providing a Streptococcus thermophilus strain, having a ratio LacS_{pH4.5} over LacZ_{pH4.5} which is less than 5, wherein LacS_{pH4.5} represents the activity of lactose importation of the LacS permease calculated by assay A at pH 4.5, and LacZ_{pH4.5} represents the activity of lactose hydrolysis of the beta-galactosidase calculated by assay B at pH 4.5; b) replacing the allele of the lacZ gene of said Streptococcus thermophilus strain of step a) with a polynucleotide of the invention, or replacing a part of the allele of the lacZ gene of said Streptococcus thermophilus strain of step a) by the corresponding polynucleotide according to the invention, or modifying the sequence of the lacZ gene of said Streptococcus thermophilus strain of step a) to have a lacZ^{FS} allele with the same sequence as a polynucleotide of the invention; and c) recovering the Streptococcus thermophilus strain(s) with a full STOP phenotype when used to ferment milk by assay C.

Thus, the invention is directed to a method to prepare a Streptococcus thermophilus strain with a full STOP phenotype, comprising: a) providing a Streptococcus thermophilus strain, having a ratio of the activity of lactose importation of the LacS permease calculated by assay A at pH 4.5 over the activity of lactose hydrolysis of the beta-galactosidase calculated by assay B at pH 4.5 (ratio LacS_{pH4.5} over LacZ_{pH4.5}) which is less than 5; b) replacing the allele of the lacZ gene of said Streptococcus thermophilus strain of step a) with a polynucleotide of the invention or replacing a part of the allele of the lacZ gene of said Streptococcus thermophilus strain of step a) by the corresponding polynucleotide according to the invention, or modifying the sequence of the lacZ gene of said Streptococcus thermophilus strain of step a) to have a lacZ^{FS} allele with the same sequence as a polynucleotide of the invention; and c) recovering the Streptococcus thermophilus strain(s) with a full STOP phenotype when used to ferment milk by assay C.

In one aspect, the invention is directed to a method to identify a lacZ^{FS} allele encoding a β-galactosidase^{FS}, comprising: a) inserting the lacZ allele to be tested in lieu of the allele of the lacZ gene of the strain DGCC715 (deposited at the DSMZ on Feb. 12, 2019 under the accession number DSM33036), to obtain a DGCC715-derivative; and b) determining the activity of lactose importation of the LacS permease by assay A at pH 4.5 (LacS_{pH4.5}) and the activity of lactose hydrolysis of the beta-galactosidase by assay B at pH 4.5 (LacZ_{pH4.5}); wherein a ratio LacS_{pH4.5} over LacZ_{pH4.5} which is more than 8 is indicative of a lacZ allele which is a lacZ^{FS} allele encoding a β-galactosidase^{FS}.

### DETAILED DESCRIPTION OF THE INVENTION

### General Definitions

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be used by reference to the specification as a whole. Accordingly, the terms defined immediately below are more fully defined by reference to the specification as a whole.

As used herein, the term “polynucleotide” is synonymous with the term “nucleotide sequence” and/or the term “nucleic acid sequence”. Unless otherwise indicated, any nucleic acid sequences are written left to right in 5 to 3′ orientation.

The term “protein”, as used herein, includes proteins, polypeptides, and peptides. As used herein, the term “amino acid sequence” is synonymous with the term “protein”. In the present disclosure and claims, the name of the amino acid, the conventional three-letter code or the conventional one-letter code for amino acid residues is used. It is also understood that a protein may be coded for by more than one nucleotide sequence due to the degeneracy of the genetic code. Unless otherwise indicated, any amino acid sequences are written left to right in amino to carboxy orientation.

In the present invention, a specific numbering of amino acid residue positions in the beta-galactosidase may be employed. By alignment of the amino acid sequence of a sample beta-galactosidase with the beta-galactosidase of SEQ ID NO: 2 it is possible to allot a number to an amino acid residue position in said sample beta-galactosidase which corresponds to the amino acid residue position or numbering of the amino acid sequence shown in SEQ ID NO: 2 of the present invention.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The present invention surprisingly found that mutations modifying the flux of lactose can be used to design Streptococcus thermophilus strains, which can be used to produce fermented milk not undergoing post-acidification when stored at fermentation temperature.

In an aspect, the present invention provides a method to identify a lacZ^{FS} allele encoding a β-galactosidase^{FS}, comprising:
a) inserting the lacZ allele to be tested in lieu of the allele of the lacZ gene of the strain DGCC715, to obtain a DGCC715-derivative; and
b) determining the activity of lactose importation of the LacS permease by assay A at pH 4.5 (LacS_{pH4.5}) and the activity of lactose hydrolysis of the beta-galactosidase by assay B at pH 4.5 (LacZ_{pH4.5}) in the DGCC715-derivative of step a);
   wherein a ratio LacS_{pH4.5} over LacZ_{pH4.5} which is more than 8 is indicative of a lacZ allele which is a lacZ^{FS} allele encoding a β-galactosidase^{FS}

In an embodiment, the method further comprises determining the activity of lactose hydrolysis of the beta-galactosidase by assay B at pH 6 (LacZ_{pH6}) in the DGCC715-derivative, and wherein a ratio LacS_{pH4.5} over LacZ_{pH4.5} which is more than 8 and a LacZ_{pH6} which is at least 7.10⁻⁸ mol/(mg of total protein extract·min) are indicative of a lacZ allele which is a lacZ^{FS} allele encoding a β-galactosidase^{FS}

As used herein, the expression “an allele of the lacZ gene” means the version of the lacZ gene found in a particular Streptococcus thermophilus strain. As for most of the bacterial genes, the nucleotide sequence of a gene can vary, and alleles represent the different sequences of the same gene.

The lacZ gene of a Streptococcus thermophilus strain is understood herein as the nucleotide sequence encoding a beta-galactosidase, located downstream of the lacS gene encoding the lactose permease LacS, within the lac operon [Schroeder C J et al., J Gen Microbiol. 1991 February; 137(2):369-80]. The word “beta-galactosidase” is used herein interchangeably with the word “β-galactosidase”.

An example of allele of the lacZ gene of Streptococcus thermophilus is the allele of the lacZ gene of the DGCC715 strain (DSM33036) which is as set forth in SEQ ID NO:1. This allele as defined in SEQ ID NO:1 encodes a β-galactosidase as set forth in SEQ ID NO:2.

An example of allele of the lacS gene of Streptococcus thermophilus is the allele of the lacS gene of the DGCC715 strain, which is as set forth in SEQ ID NO:30. This allele as defined in SEQ ID NO:30 encodes a lactose permease LacS as set forth in SEQ ID NO:31.

lacZ^{FS} Alleles Encoding β-Galactosidase^{FS}

The inventors have shown that some of these lacZ alleles encode a β-galactosidase, the activity of which is largely reduced but not null at pH 4.5 (as determined by assay B), when inserted in lieu of the allele of the lacZ gene (SEQ ID NO:1) of the DGCC715 strain. By “β-galactosidase activity not null at pH 4.5”, it is meant that the β-galactosidase activity at pH 4.5 (LacZ_{pH4.5}) is detectable when determined by assay B as described herein.

As shown in examples 4 and 5 below, the β-galactosidase activity in Streptococcus thermophilus strains is highly variable from a strain to another, such that it is not technically pertinent to refer to β-galactosidase activity without having any reference value or without having any reference strain. Moreover, and as shown in example 6, the reduction of the β-galactosidase activity at pH 4.5 in a DGCC715-derivative strain bearing a lacZ^{FS} allele, as compared to the DGCC715 strain, goes together with an increase of the LacS activity (as determined by assay A). Altogether, these results have led the inventors to characterize the reduction of the β-galactosidase at pH 4.5 by a robust and reproducible parameter, which is the ratio of the activity of lactose importation of the LacS permease calculated by assay A at pH 4.5 over the activity of lactose hydrolysis of the beta-galactosidase calculated by assay B at pH 4.5 (ratio LacS_{pH4.5} over LacZ_{pH4.5}). Thus, the inventors have shown that one of these lacZ alleles leads to a ratio LacS_{pH4.5} over LacZ_{pH4.5} of more than 8, when inserted in lieu of the allele of the lacZ gene (SEQ ID NO:1) of the DGCC715 strain. These lacZ alleles are defined herein as “lacZ^{FS} alleles”. The protein encoded by these lacZ^{FS} alleles is referred herein as “β-galactosidase^{FS}”. In other words, a lacZ^{FS} allele increases the ratio of the activity of lactose importation of the LacS permease calculated by assay A at pH 4.5 over the activity of lactose hydrolysis of the beta-galactosidase calculated by assay B at pH 4.5 (ratio LacS_{pH4.5} over LacZ_{pH4.5}) above 8 in a DGCC715 derivative, said DGCC715 derivative being a strain DGCC715 (DSM33036), into which its lacZ gene was replaced by said polynucleotide encoding a β-galactosidase^{FS}; as defined within this application, the “increase” of the ratio LacS_{pH4.5} over LacZ_{pH4.5} in a DGCC715 derivative is determined compared to the ratio LacS_{pH4.5} over LacZ_{pH4.5} of the strain DGCC715 (DSM33036).

Thus, any lacZ^{FS} allele (encoding a β-galactosidase^{FS}) leading to a ratio LacS_{pH4.5} over LacZ_{pH4.5} of more than 8 (as defined herein) in a DGCC715-derivative is part of the invention. Thus, any lacZ^{FS} allele (encoding a β-galactosidase^{FS}) increasing the ratio LacS_{pH4.5} over LacZ_{pH4.5} above 8 in a DGCC715-derivative as defined herein is part of the invention. In an embodiment, the lacZ^{FS} allele of the invention (encoding a β-galactosidase^{FS}) leads to a ratio LacS_{pH4.5} over LacZ_{pH4.5} which is more than 9 (as defined herein) in a DGCC715-derivative. In an embodiment, the lacZ^{FS} allele of the invention (encoding a β-galactosidase^{FS}) leads to a ratio LacS_{pH4.5} over LacZ_{pH4.5} which is more than 10 (as defined herein) in a DGCC715-derivative. In an embodiment, the lacZ^{FS} allele of the invention (encoding a β-galactosidase^{FS}) leads to a ratio LacS_{pH4.5} over LacZ_{pH4.5} which is more than 11 (as defined herein) in a DGCC715-derivative. In an embodiment, the lacZ^{FS} allele of the invention (encoding a β-galactosidase^{FS}) leads to a ratio LacS_{pH4.5} over LacZ_{pH4.5} which is more than 12 (as defined herein) in a DGCC715-derivative. In an embodiment, the lacZ^{FS} allele of the invention (encoding a β-galactosidase^{FS}) leads to a ratio LacS_{pH4.5} over LacZ_{pH4.5} (as defined herein) in a DGCC715-derivative which is selected from the group consisting of more than 9, more than 10, more than 11 and more than 12. Thus, the lacZ^{FS} allele of the invention (encoding a β-galactosidase^{FS}) increases the ratio LacS_{pH4.5} over LacZ_{pH4.5}, in a DGCC715-derivative as defined herein, above a value selected from the group consisting of above 9, above 10, above 11 and above 12.

As mentioned elsewhere, the S-galactosidase activity at pH 4.5 (LacZ_{pH4.5}) is not null, i.e., detectable when determined by assay B; in an embodiment, and in combination with any minimal value regarding the ratio LacS_{pH4.5} over LacZ_{pH4.5} as defined herein, the lacZ^{FS} allele of the invention (encoding a S-galactosidase^{FS}) leads to a ratio LacS_{pH4.5} over LacZ_{pH4.5} (as defined herein) in a DGCC715-derivative which is less than 100 (or increases the ratio LacS_{pH4.5} over LacZ_{pH4.5}, in a DGCC715-derivative, to less than 100).

In an embodiment, the lacZ^{FS} allele as defined herein is further characterized (in addition to the ratio LacS_{pH4.5} over LacZ_{pH4.5}) by the fact that it encodes a S-galactosidase^{FS} the activity of which is at least 7.10⁻⁸ mol/(mg of total protein extract·min) at pH 6 (as determined by assay B) (LacZ_{pH6}), when said lacZ^{FS} allele is inserted in lieu of the allele of the lacZ gene of DGCC715 strain. Thus, the lacZ^{FS} allele as defined herein is further characterized (in addition to the ratio LacS_{pH4.5} over LacZ_{pH4.5}) by the fact that it encodes a S-galactosidase^{FS} the activity of which is at least 7.10⁻⁸ mol/(mg of total protein extract·min) at pH 6 (as determined by assay B) (LacZ_{pH6}) in a DGCC715 derivative, said DGCC715 derivative being a strain DGCC715 into which its lacZ gene was replaced by said lacZ^{FS} allele. In an embodiment, the lacZ^{FS} allele encodes a S-galactosidase^{FS} the activity of which is at least 8.10⁻⁸ mol/(mg of total protein extract·min) at pH 6 (LacZ_{pH6}). In an embodiment, the lacZ^{FS} allele encodes a R-galactosidase^{FS} the activity of which is at least 9.10⁻⁸ mol/(mg of total protein extract·min) at pH 6 (LacZ_{pH6}). In an embodiment, the lacZ^{FS} allele encodes a β-galactosidase^{FS} the activity of which is at least 1.10⁻⁷ mol/(mg of total protein extract·min) at pH 6 (LacZ_{pH6}). In an embodiment, the lacZ^{FS} allele as defined herein is further characterized (in addition to the ratio LacS_{pH4.5} over LacZ_{pH4.5}) by the fact that it encodes a S-galactosidase^{FS} the activity of which is selected from the group consisting of at least 7.10⁻⁸, at least 8.10⁻⁸, at least 9.10⁻⁸ and at least 1.10⁻⁷ mol/(mg of total protein extract·min) at pH 6 (as determined by assay B) (LacZ_{pH6}), when said lacZ^{FS} allele is inserted in lieu of the allele of the lacZ gene of DGCC715 strain (i.e., in a DGCC715 derivative, said DGCC715 derivative being a strain DGCC715 into which its lacZ gene was replaced by said lacZ^{FS} allele).

Thus, in an embodiment, any lacZ^{FS} allele (encoding a β-galactosidase^{FS}) leading to a ratio LacS_{pH4.5} over LacZ_{pH4.5} of more than 8 (as defined herein) and leading to a LacZ_{pH6} of at least 7.10⁻⁸ mol/(mg of total protein extract·min) (as defined herein), in a DGCC715-derivative, is part of the invention. In an embodiment, the lacZ^{FS} allele of the invention (encoding a β-galactosidase^{FS}) leads to a ratio LacS_{pH4.5} over LacZ_{pH4.5} which is selected from the group consisting of more than 9, more than 10, more than 11 and more than 12 (as defined herein) in a DGCC715-derivative, and leads to a LacZ_{pH6} selected from the group consisting of at least 7.10⁻⁸, at least 8.10⁻⁸, at least 9.10⁻⁸ and at least 1.10⁻⁷ mol/(mg of total protein extract·min) (as determined by assay B) in said DGCC715-derivative. In an embodiment, the lacZ^{FS} allele of the invention (encoding a β-galactosidase^{FS}) leads to a ratio LacS_{pH4.5} over LacZ_{pH4.5} (as defined herein) in a DGCC715-derivative which is less than 100. Thus, any lacZ^{FS} allele (encoding a β-galactosidase^{FS}) increasing the ratio LacS_{pH4.5} over LacZ_{pH4.5} above 8 (compared to the ratio LacS_{pH4.5} over LacZ_{pH4.5} of the strain DGCC715) and leading to a LacZ_{pH6} of at least 7.10⁻⁸ mol/(mg of total protein extract·min) (as defined herein), in a DGCC715-derivative, is part of the invention. In an embodiment, the lacZ^{FS} allele of the invention (encoding a β-galactosidase^{FS}) increases the ratio LacS_{pH4.5} over LacZ_{pH4.5} above a value which is selected from the group consisting of above 9, above 10, above 11 and above 12 (as defined herein) in a DGCC715-derivative, and leads to a LacZ_{pH6} selected from the group consisting of at least 7.10⁻⁸, at least 8.10⁻⁸, at least 9.10⁻⁸ and at least 1.10⁻⁷ mol/(mg of total protein extract·min) (as determined by assay B) in said DGCC715-derivative. In an embodiment, the lacZ^{FS} allele of the invention (encoding a β-galactosidase^{FS}) increases the ratio LacS_{pH4.5} over LacZ_{pH4.5} (as defined herein) in a DGCC715-derivative to less than 100.

Non-limitative examples of β-galactosidase^{FS} are disclosed below.

It is noteworthy that in the present invention, the LacS and LacZ activity (at pH 4.5 and at pH 6) are calculated by the assay A and the assay B respectively, as described herein.

A lacZ allele which, when inserted in lieu of the allele of the lacZ gene of DGCC715 strain does not lead to a ratio LacS_{pH4.5} over LacZ_{pH4.5} (as defined herein) of more than 8 is not considered to be a lacZ^{FS} allele according to the invention. In other words, a lacZ allele which, does not increase the ratio LacS_{pH4.5} over LacZ_{pH4.5} (as defined herein) above 8 in a DGCC715 derivative is not considered to be a lacZ^{FS} allele according to the invention, said DGCC715 derivative being a strain DGCC715 into which its lacZ gene was replaced by said lacZ allele.

### LacS Activity, LacZ Activity and Ratio

The invention relies on the determination of activity of lactose importation of the LacS permease and/or the determination of the activity of lactose hydrolysis of the beta-galactosidase, at particular pHs (pH 4.5 and/or pH 6). These activities are determined in a particular strain, such as for example in the DGCC715 strain or in a DGCC715-derivative as defined herein.

The activity of lactose importation of the LacS permease at a particular pH (pH X) is referred herein as “LacS_{pHx}”. In an embodiment, this activity is determined at pH 4.5 (LacS_{pH4.5}). In an embodiment, this activity is determined at pH 6 (LacS_{pH6}). In a particular embodiment, the activity of lactose importation of the LacS permease is determined at a particular pH (such as pH 4.5 or pH 6) by assay A.

The activity of lactose hydrolysis of the beta-galactosidase at a particular pH (pH X) is referred herein as “LacZ_{pHx}”. In an embodiment, this activity is determined at pH 4.5 (LacZ_{pH4.5}). In an embodiment, this activity is determined at pH 6 (LacZ_{pH6}). In a particular embodiment, the activity of lactose hydrolysis of the beta-galactosidase is determined at a particular pH (such as pH 4.5 or pH 6) by assay B.

One way to determine the ratio LacS_{pH4.5} over LacZ_{pH4.5} for the identification of the lacZ^{FS} allele of the invention, is to determine the activity of lactose importation of the LacS permease at pH4.5 in a DGCC715 strain in which the allele of its lacZ gene has been replaced with a lacZ allele to be tested (called herein “DGCC715-derivative”) and to determine the activity of lactose hydrolysis of the beta-galactosidase at pH 4.5 in the same DGCC715-derivative, and to calculate the ratio of both activities.

### Assay A (LacS Activity)

Streptococcus thermophilus strains were grown on M17 media containing 30 g/L of sucrose as sole carbon source overnight at 37° C. When cells reached the stationary phase, they were transferred (at 0.05 uDO/mL) in 1 volume of M17 media containing 30 g/L of lactose as sole carbon source and they were incubated for 2 hours at 42° C. Strain cultures were centrifuged at room temperature (3500 g), the supernatant was removed and cells were resuspended in 0.5 volume of 4% (w/v) glycerophosphate. This washing step was applied twice. 1.8 mL of cell suspension in 4% glycerophosphate were incubated for 2 minutes at 42° C. Then, 0.2 mL of lactose solution (70 g/L of lactose+0.1 M potassium phosphate buffer) was added [the lactose solution pH was previously adjusted at pH 4.5 or at pH 6, depending on the measurement needed]. The mix was incubated for 3 additional minutes at 42° C. The reaction was blocked by filtrating on 0.22 μm filter in order to remove cells. Then, the lactose in the filtrated solution was assayed on an HPLC using the following protocol. The solution was diluted 10-fold in water and 10 μL were injected on an Agilent 1200 HPLC (high-performance-liquid-chromatography). The elution was done in isocratic mode with pure water at 0.6 mL/min. Molecules were separated in 40 min onto a Pb²⁺ ion exchange column (SP-0810 Shodex® 300 mm×8 mm×7 μm) column. Sugars were detected with refractometer. Quantification was performed by external calibration.

The activity of lactose importation of the LacS permease is calculated as follows:

$$LacS activity = ( {[ lactose ]}_{initial} - {[ lactose ]}_{3 min} ) / ( DO \times time ) , expressed in μ mol / ( uDO . min ) ,$$

wherein:
[lactose]ᵢₙᵢₜᵢₐₗ is the initial concentration in μmol/mL
[lactose]₃ₘᵢₙ is the concentration in μmol/mL after 3 minutes at 42° C.
DO is the bacterial density in uDO/mL
time is the experiment duration in minutes (in the present case, 3 minutes).

### Assay B (LacZ Activity)

A fresh overnight culture of the Streptococcus thermophilus strain to be assayed in M17 containing 30 g/L lactose was obtained and used to inoculate at 1% (v/v) 10 ml of fresh M17 containing 30 g/L lactose. Cells were harvested by centrifugation (6000 g, 10 min, 4° C.) after 3 hours of growth on M17 containing 30 g/L lactose at 42° C., washed in 1.5 ml of cold lysis buffer (KPO4 0.1 M), and resuspended in 300 μl of cold lysis buffer. EDTA-free protease inhibitors “cOmplete™” (Roche, supplier reference 04693132001) was added to the lysis buffer as described by the supplier. Cells were disrupted by the addition of 100 mg glass beads (150-212 μm, Sigma G1145) to 250 μl of resuspended cells and oscillation at a frequency of 30 cycles/s for 6 min in a MM200 oscillating mill (Retsch, Haan, Germany). Cell debris and glass beads were removed by centrifugation (14000 g, 15 min, 4° C.), and the supernatant was transferred into a clean 1.5 mL centrifuge tube kept on ice. Total protein content was determined by using the FLUKA Protein Quantification Kit-Rapid (ref 51254). The beta-galactosidase activity in the cell extracts was determined spectrophotometrically by a monitoring of the hydrolysis of O-nitro-Phenol-Beta-Galactoside (ONPG) into galactose and O-nitro-phenol (ONP). Twenty μL of the cell extract were mixed with 135 μL of React Buffer (NaPO₄ 100 mM; KCl 10 mM; MgSO₄ 1 mM; ONPG 3 mM+Beta Mercapto Ethanol 60 mM, pH=6). The production of ONP leads to a yellow color into the tube. When the yellow color was appearing, the reaction was blocked by adding 250 μL of Stopping buffer (Na₂CO₃ 1 M).

The optical density at 420 nm was recorded using a Synergy HT multi-detection microplate reader (BIO-TEK). One unit of beta-galactosidase corresponds to the amount of enzyme that catalyzes the production of 1 μmole ONP per minute under the assay conditions. Beta-galactosidase activity was calculated as follows:

LacZ activity=dOD×V/[dt×l×ε×Qprot], expressed in mol/(mg of total protein extract·min), wherein:
dOD is the variation of optical density (OD) at 420 nm between the blank and the tested sample
V is the volume of the reaction in which the optical density is measured (herein 250 μL)
dt=represent the duration in minutes between the addition of the 20 μL of bacterial extract and the addition of the 250 μL stopping buffer
l=optical path length (herein 0.73 cm)
ε=molar attenuation coefficient of ONP (herein 4500 cm²/μmol)
Qprot=quantity of protein in the cuvette (in mg)

### Ratio Calculation

Once the LacS and LacZ activities have been calculated as defined herein, the ratio of the activities LacS_{pHX} over LacZ_{pHX}, is calculated as follows: [LacS_{pHX} as defined herein/LacZ_{pHX} as defined herein]×10⁻⁶.

It is noteworthy that when a ratio LacS_{pHX} over LacZ_{pHX} is mentioned, both the LacS and LacZ activities are calculated in the same strain, in particular in the same DGCC715-derivative.

lacZ Variant Allele Encoding a β-Galactosidase Variant

A lacZ allele, which 1) encodes β-galactosidase the sequence of which has at least 95% identity with SEQ ID NO:2, and 2) leads to a ratio LacS_{pH4.5} over LacZ_{pH4.5} (as defined herein) of less than 5, when inserted in lieu of the allele of the lacZ gene of DGCC715 strain, is referred herein as a lacZ variant allele (encoding a β-galactosidase variant). In other words, a lacZ allele, which 1) encodes β-galactosidase the sequence of which has at least 95% identity with SEQ ID NO:2, and 2) does not increase the ratio LacS_{pH4.5} over LacZ_{pH4.5} (as defined herein) to 5 or more than 5, in a DGCC715 derivative, is referred herein as a lacZ variant allele (encoding a β-galactosidase variant), said DGCC715 derivative being a strain DGCC715 into which its lacZ gene was replaced by said lacZ variant allele; as previously mentioned, the “increase” of the ratio LacS_{pH4.5} over LacZ_{pH4.5} in a DGCC715 derivative is determined compared to the ratio LacS_{pH4.5} over LacZ_{pH4.5} of the strain DGCC715 (DSM33036). The expression “β-galactosidase variant” is used interchangeably with the expression “β-galactosidase variant having at least 95% identity with SEQ ID NO:2”.

In an embodiment, the lacZ variant allele, when inserted in lieu of the allele of the lacZ gene of DGCC715 strain, leads to a ratio LacS_{pH4.5} over LacZ_{pH4.5} (as defined herein) of less than 4 (or does not increase the ratio LacS_{pH4.5} over LacZ_{pH4.5} to 4 or more than 4 in a DGCC715 derivative as defined herein). In an embodiment, the lacZ variant allele, when inserted in lieu of the allele of the lacZ gene of DGCC715 strain, leads to a ratio LacS_{pH4.5} over LacZ_{pH4.5} (as defined herein) of less than 3 (or does not increase the ratio LacS_{pH4.5} over LacZ_{pH4.5} to 3 or more than 3 in a DGCC715 derivative as defined herein).

In combination with any of the embodiments directed to the ratio LacS_{pH4.5} over LacZ_{pH4.5} above, a lacZ variant allele is also defined as encoding a β-galactosidase variant, the sequence of which is at least 95% identical to SEQ ID NO:2. By “at least 95% identical to SEQ ID NO:2”, it is meant at least 95%, at least 96%, at least 97%, at least 98% or at least 99%. In an embodiment, a β-galactosidase variant (encoded by a lacZ variant allele) has a sequence which is at least 96% identical to SEQ ID NO:2. In an embodiment, a β-galactosidase variant (encoded by a lacZ variant allele) has a sequence which is at least 97% identical to SEQ ID NO:2. In an embodiment, a β-galactosidase variant (encoded by a lacZ variant allele) has a sequence which is at least 98% identical to SEQ ID NO:2. In an embodiment, a β-galactosidase variant (encoded by a lacZ variant allele) has a sequence which is at least 99% identical to SEQ ID NO:2.

In an embodiment, in combination with the percentage of identity, the size of the β-galactosidase variant is the same as the β-galactosidase protein as defined in SEQ ID NO:2 (1026 amino acid residues); thus, in an embodiment, a lacZ variant allele is additionally defined as encoding a 1026-amino acid β-galactosidase variant.

In an embodiment, a lacZ variant allele is defined herein as:
1) encoding a β-galactosidase variant, the sequence of which is at least 95%, at least 96%, at least 97%, at least 98% or at least 99% identical to SEQ ID NO:2; and
2) when inserted in lieu of the allele of the lacZ gene of the DGCC715 strain, leads to a ratio LacS_{pH4.5} over LacZ_{pH4.5} (as defined herein) which is less than 5, less than 4 or less than 3.

Thus, a lacZ variant allele is defined herein as:
1) encoding a β-galactosidase variant, the sequence of which is at least 95%, at least 96%, at least 97%, at least 98% or at least 99% identical to SEQ ID NO:2; and
2) not increasing the ratio LacS_{pH4.5} over LacZ_{pH4.5} to 5 or more than 5, to 4 or more than 4 or to 3 or more than 3 in a DGCC715 derivative as defined herein.

Non-limitative examples of β-galactosidase variants are disclosed in Table 2, and their sequence is as defined in SEQ ID Nos 6, 9, 12, 15, 18, 21, 24 and 27.

Replacement of the Allele of the lacZ Gene of a Streptococcus thermophilus Strain (in Particular of the DGCC715 Strain)

The replacement of the allele of the lacZ gene of a particular Streptococcus thermophilus strain by a lacZ allele to be tested is carried out using conventional techniques in molecular biology and is within the capabilities of a person of ordinary skill in the art. Generally speaking, suitable routine methods include replacement via homologous recombination.

The expression “lacZ allele inserted in lieu of the allele of the lacZ gene” is synonymous to the expression “the allele of the lacZ gene is replaced by a lacZ allele to be tested”. The expression “lacZ^{FS} allele inserted in lieu of the allele of the lacZ gene” is synonymous to the expression “the allele of the lacZ gene is replaced by a lacZ^{FS} allele”.

Replaced (or inserted in lieu) means that the sequence of the β-galactosidase encoded by the lacZ allele to be inserted (the lacZ allele to be tested) is different from the sequence of the β-galactosidase encoded by the allele of the lacZ gene of the Streptococcus thermophilus strain. Thus, replaced (or inserted in lieu) means that the coding sequence of the lacZ gene of the Streptococcus thermophilus strain (from the 1^{st} nucleotide of the start codon to the last nucleotide of the stop codon) is replaced by the corresponding coding sequence of the lacZ allele to be tested.

In the case of the DGCC715 strain, replaced (or inserted in lieu) means that the sequence of the β-galactosidase protein encoded by the lacZ allele to be inserted (the lacZ allele to be tested) is different from the sequence of the β-galactosidase encoded by the lacZ gene of the DGCC715 strain. Thus, replaced (or inserted in lieu) means that the coding sequence of the lacZ gene of the DGCC715 strain (from the 1^{st} nucleotide of the start codon to the last nucleotide of the stop codon, i.e., nucleotides 1 to 3081 of SEQ ID NO:1) is replaced by the corresponding coding sequence of the lacZ allele to be tested. A DGCC715 strain, the lacZ gene of which has been replaced by a lacZ allele to be tested (such as a lacZ^{FS} allele or a lacZ variant allele), is defined herein as a “DGCC715-derivative”.

### DGCC715 Strain

The Streptococcus thermophilus DGCC715 strain has been deposited by DuPont Nutrition Biosciences ApS under the Budapest Treaty at the Leibniz-Institut DSMZ-Deutsche Sammlung von Mikroorganismen und Zellkulturen, GmbH (Inhoffenstr. 7B, D-38124 Braunschweig), on Feb. 12, 2019 and have received the deposit number DSM33036. Conditions for culturing this strain are provided in the examples part. The applicant requests that a sample of the deposited micro-organism stated herein may only be made available to an expert, until the date on which the patent is granted.

The expressions “DGCC715 strain” and “DGCC715-derivative” are used interchangeably with the expressions “DSM33036 strain” and “DSM33036-derivative” respectively.

To Generate a lacZ Allele to be Tested (Including a lacZ^{FS} Allele)

lacZ alleles to be tested (in particular lacZ^{FS} alleles) can be generated by random or directed mutagenesis, starting from a lacZ allele which is not a lacZ^{FS} allele, in particular starting from a lacZ allele encoding the β-galactosidase as defined in SEQ ID NO:2 (such as SEQ ID NO:1) or starting from a lacZ variant allele as defined herein. In an embodiment, lacZ alleles to be tested (in particular lacZ^{FS} alleles) are generated by random mutagenesis. In another embodiment, lacZ alleles to be tested (in particular lacZ^{FS} alleles) can be generated by directed mutagenesis. Suitable mutagenesis protocols for random or directed mutagenesis are well known and described in the literature.

The lacZ alleles to be tested thus generated can be screened using the method to identify a lacZ^{FS} allele as defined herein.

### Sequences of β-Galactosidase^{FS} Proteins

The lacZ^{FS} allele of the invention—as part of a polynucleotide of the invention or contained in the lactic acid bacterium of the invention—can be defined, in addition to lead to a ratio LacS_{pH4.5} over LacZ_{pH4.5} of more than 8 (as defined herein) (or to increase the ratio LacS_{pH4.5} over LacZ_{pH4.5} to more than 8) and optionally to lead to a LacZ_{pH6} of at least 7.10⁻⁸ mol/(mg of total protein extract·min) (as defined herein), by its nucleotide sequence or by the amino acid sequence of the β-galactosidase it encodes.

In an embodiment, the lacZ^{FS} allele as defined herein encodes a β-galactosidase^{FS}, the sequence of which is different from SEQ ID NO:2. In an embodiment, the lacZ^{FS} allele as defined herein—as part of a polynucleotide of the invention or contained in the lactic acid bacterium of the invention—is defined by the fact that it leads to a ratio LacS_{pH4.5} over LacZ_{pH4.5} of more than 8 (as defined herein) (or increases the ratio LacS_{pH4.5} over LacZ_{pH4.5} to more than 8), and optionally to a LacZ_{pH6} of at least 7.10⁻⁸ mol/(mg of total protein extract·min) (as defined herein), in a DGCC715-derivative, and that it encodes a β-galactosidase^{FS}, the sequence of which is different from SEQ ID NO:2. Particular embodiments regarding the ratio LacS_{pH4.5} over LacZ_{pH4.5} and the LacZ_{pH6} described elsewhere in this application apply similarly in the current context.

In an embodiment, the lacZ^{FS} allele encodes a β-galactosidase^{FS} comprising an amino-acid suppression (i.e., the suppression of one or more an amino acids), an amino-acid addition (i.e., the addition of one or more an amino acids), an amino-acid substitution (i.e., the substitution of one or more an amino acids) or an amino-acid suppression and addition (i.e., the suppression and addition of one or more an amino acids), relative to a β-galactosidase selected from the group consisting of:
a) a β-galactosidase having an amino-acid sequence as defined in SEQ ID NO:2; and
b) a β-galactosidase variant protein as defined herein having at least 95% identity with SEQ ID NO:2. A β-galactosidase variant protein as defined herein is encoded by a lacZ variant allele, which when inserted in lieu of the allele of the lacZ gene of the DGCC715 strain, leads to a ratio LacS_{pH4.5} over LacZ_{pH4.5} which is less than 5 (as defined herein) (or does not increase the ratio LacS_{pH4.5} over LacZ_{pH4.5} to 5 or more than 5 in a in a DGCC715 derivative as defined herein). Particular embodiments regarding the ratio LacS_{pH4.5} over LacZ_{pH4.5}, percentage of identity and size described elsewhere in this application within the context of the lacZ variant allele apply similarly in the current context.

In an embodiment, the lacZ^{FS} allele encodes a β-galactosidase^{FS} comprising an amino acid suppression, relative to a β-galactosidase selected from the group consisting of a) a β-galactosidase having an amino acid sequence as defined in SEQ ID NO:2 and b) a β-galactosidase variant as defined herein having at least 95% identity with SEQ ID NO:2; in a particular embodiment, the β-galactosidase^{FS} is characterized by the suppression of at least one amino acid, in particular by the suppression of 1, 2, 3, 4 or 5 amino acids. In a particular embodiment, the β-galactosidase^{FS} is characterized by the suppression of one amino acid. In a particular embodiment, the β-galactosidase^{FS} is characterized by the suppression of 2, 3, 4 or 5 amino acids. In a particular embodiment, the β-galactosidase^{FS} is characterized by the suppression of 2, 3, 4 or 5 consecutive amino acids.

In an embodiment, the lacZ^{FS} allele encodes a β-galactosidase^{FS} comprising an amino acid addition, relative to a β-galactosidase selected from the group consisting of a) a β-galactosidase having an amino acid sequence as defined in SEQ ID NO:2 and b) a β-galactosidase variant as defined herein having at least 95% identity with SEQ ID NO:2; in a particular embodiment, the β-galactosidase^{FS} is characterized by the addition of at least one amino acid, in particular by the addition of 1, 2, 3, 4 or 5 amino acids. In a particular embodiment, the β-galactosidase^{FS} is characterized by the addition of one amino acid. In a particular embodiment, the β-galactosidase^{FS} is characterized by the addition of 2, 3, 4 or 5 amino acids. In a particular embodiment, the β-galactosidase^{FS} is characterized by the addition of 2, 3, 4 or 5 consecutive amino acids.

In an embodiment, the lacZ^{FS} allele encodes a β-galactosidase^{FS} comprising an amino acid substitution relative to a β-galactosidase selected from the group consisting of a) a β-galactosidase having an amino acid sequence as defined in SEQ ID NO:2 and b) a β-galactosidase variant as defined herein having at least 95% identity with SEQ ID NO:2; in a particular embodiment, the β-galactosidase^{FS} is characterized by the substitution of at least one amino acid, in particular by the substitution of 1, 2, 3, 4 or 5 amino acids. In a particular embodiment, the β-galactosidase^{FS} is characterized by the substitution of one amino acid. In a particular embodiment, the β-galactosidase^{FS} is characterized by the substitution of 2, 3, 4 or 5 amino acids. In a particular embodiment, the β-galactosidase^{FS} is 1026 amino acids in length.

In an embodiment, the lacZ^{FS} allele encodes a β-galactosidase^{FS}, wherein the sequence of said β-galactosidase^{FS} does not comprise an arginine at position 354, wherein the amino acid sequence set forth in SEQ ID NO:2 is used for numbering.

In an embodiment, the lacZ^{FS} allele encodes a β-galactosidase^{FS}, wherein the sequence of said β-galactosidase^{FS} does not comprise an amino acid residue selected from the group consisting of arginine, histidine, glutamine and lysine at position 354, wherein the amino acid sequence set forth in SEQ ID NO:2 is used for numbering. In an embodiment, the lacZ^{FS} allele encodes a β-galactosidase^{FS}, wherein the sequence of said β-galactosidase^{FS} does not comprise an amino acid residue selected from the group consisting of arginine, histidine, glutamine, lysine, glutamic acid and asparagine at position 354, wherein the amino acid sequence set forth in SEQ ID NO:2 is used for numbering

In an embodiment, the lacZ^{FS} allele encodes a β-galactosidase^{FS} comprising a cysteine or an equivalent amino acid thereof at position 354, wherein the amino acid sequence set forth in SEQ ID NO:2 is used for numbering. By “equivalent amino acid thereof”, it is meant any amino acid having similarity in polarity, charge, solubility, hydrophobicity, hydrophilicity, and/or the amphipathic nature of the residues, as long as the lacZ^{FS} allele encoding this R-galactosidase^{FS}, leads to a ratio LacS_{pH4.5} over LacZ_{pH4.5} of more than 8 (as defined herein) and optionally leads to a LacZ_{pH6} of at least 7.10⁻⁸ mol/(mg of total protein extract·min) (as defined herein), when inserted in lieu of the allele of the lacZ gene of the DGCC715 strain. In an embodiment, the lacZ^{FS} allele encodes a β-galactosidase^{FS} comprising an amino acid residue selected from the group consisting of cysteine, alanine and serine at position 354, wherein the amino acid sequence set forth in SEQ ID NO:2 is used for numbering

In an embodiment, the lacZ^{FS} allele encodes a β-galactosidase^{FS} comprising a cysteine at position 354, wherein the amino acid sequence set forth in SEQ ID NO:2 is used for numbering.

In a particular embodiment of any of these embodiments, the β-galactosidase^{FS} is 1026 amino acids in length.

In an embodiment, the lacZ^{FS} allele of the invention encodes a S-galactosidase^{FS}, the sequence of which is at least 95% identical to, but different from, SEQ ID NO:2.

In an embodiment, the lacZ^{FS} allele encodes a β-galactosidase^{FS}, the sequence of which is at least 95% identical to, but different from, SEQ ID NO:2, and does not comprise an arginine at position 354, wherein the amino acid sequence set forth in SEQ ID NO:2 is used for numbering.

In an embodiment, the lacZ^{FS} allele encodes a β-galactosidase^{FS}, the sequence of which is at least 95% identical to, but different from, SEQ ID NO:2, and does not comprise an amino acid residue selected from the group consisting of arginine, histidine, glutamine and lysine at position 354, wherein the amino acid sequence set forth in SEQ ID NO:2 is used for numbering.

In an embodiment, the lacZ^{FS} allele encodes a β-galactosidase^{FS}, the sequence of which is at least 95% identical to, but different from, SEQ ID NO:2, and does not comprise an amino acid residue selected from the group consisting of arginine, histidine, glutamine, lysine, glutamic acid and asparagine at position 354, wherein the amino acid sequence set forth in SEQ ID NO:2 is used for numbering

In an embodiment, the lacZ^{FS} allele encodes a β-galactosidase^{FS}, the sequence of which is at least 95% identical to, but different from, SEQ ID NO:2, and comprises a cysteine or an equivalent amino acid thereof at position 354, wherein the amino acid sequence set forth in SEQ ID NO:2 is used for numbering.

In an embodiment, the lacZ^{FS} allele encodes a β-galactosidase^{FS}, the sequence of which is at least 95% identical to, but different from, SEQ ID NO:2, and comprises an amino acid residue selected from the group consisting of cysteine, alanine and serine at position 354, wherein the amino acid sequence set forth in SEQ ID NO:2 is used for numbering

In an embodiment, the lacZ^{FS} allele encodes a β-galactosidase^{FS}, the sequence of which is at least 95% identical to, but different from, SEQ ID NO:2, and comprises a cysteine at position 354, wherein the amino acid sequence set forth in SEQ ID NO:2 is used for numbering.

In a particular embodiment of any of these embodiments, the β-galactosidase^{FS} is 1026 amino acids in length.

In an embodiment, the lacZ^{FS} allele encodes a β-galactosidase^{FS} comprising:
a) an amino acid sequence which is otherwise as defined in SEQ ID NO:2, but which does not comprise an arginine at position 354 (SEQ ID NO:5, wherein position 354 is not an arginine); or
b) an amino acid sequence which is otherwise the one of a β-galactosidase variant having at least 95% identity with SEQ ID NO:2 (β-galactosidase variant as defined herein), but which does not comprise an arginine at position 354. Non-limitative examples of β-galactosidase^{FS} are as defined in SEQ ID Nos 7, 10, 13, 16, 19, 22, 25 and 28, wherein position 354 is not an arginine.

In an embodiment, the lacZ^{FS} allele encodes a β-galactosidase^{FS} comprising:
a) an amino acid sequence which is otherwise as defined in SEQ ID NO:2, but which does not comprise an amino acid residue selected from the group consisting of arginine, histidine, glutamine and lysine at position 354 (SEQ ID NO:5, wherein position 354 is not an amino acid residue selected from the group consisting of arginine, histidine, glutamine and lysine); or
b) an amino acid sequence which is otherwise the one of a β-galactosidase variant having at least 95% identity with SEQ ID NO:2 (β-galactosidase variant as defined herein), but which does not comprise an amino acid residue selected from the group consisting of arginine, histidine, glutamine and lysine at position 354. Non-limitative examples of β-galactosidase^{FS} Non-limitative examples of β-galactosidase^{FS} are as defined in SEQ ID Nos 7, 10, 13, 16, 19, 22, 25 and 28, wherein position 354 is not an amino acid residue selected from the group consisting of arginine, histidine, glutamine and lysine.

In an embodiment, the lacZ^{FS} allele encodes a β-galactosidase^{FS} comprising:
a) an amino acid sequence which is otherwise as defined in SEQ ID NO:2, but which does not comprise an amino acid residue selected from the group consisting of arginine, histidine, glutamine, lysine, glutamic acid and asparagine at position 354 (SEQ ID NO:5, wherein position 354 is not an amino acid residue selected from the group consisting of arginine, histidine, glutamine, lysine, glutamic acid and asparagine); or
b) an amino acid sequence which is otherwise the one of a β-galactosidase variant having at least 95% identity with SEQ ID NO:2 (β-galactosidase variant as defined herein), but which does not comprise an amino acid residue selected from the group consisting of arginine, histidine, glutamine, lysine, glutamic acid and asparagine at position 354. Non-limitative examples of β-galactosidase^{FS} are as defined in SEQ ID Nos 7, 10, 13, 16, 19, 22, 25 and 28, wherein position 354 is not an amino acid residue selected from the group consisting of arginine, histidine, glutamine, lysine, glutamic acid and asparagine.

In an embodiment, the lacZ^{FS} allele encodes a β-galactosidase^{FS} comprising:
a) an amino acid sequence which is otherwise as defined in SEQ ID NO:2, but which comprises a cysteine or an equivalent amino acid thereof at position 354 (SEQ ID NO:5, wherein position 354 is a cysteine or an equivalent amino acid thereof); or
b) an amino acid sequence which is otherwise the one of a β-galactosidase variant having at least 95% identity with SEQ ID NO:2 (β-galactosidase variant as defined herein), but which comprises a cysteine or an equivalent amino acid thereof at position 354. Non-limitative examples of β-galactosidase^{FS} are as defined in SEQ ID Nos 7, 10, 13, 16, 19, 22, 25 and 28, wherein position 354 is a cysteine or an equivalent amino acid thereof.

In an embodiment, the lacZ^{FS} allele encodes a β-galactosidase^{FS} comprising:
a) an amino acid sequence which is otherwise as defined in SEQ ID NO:2, but which comprises an amino acid residue selected from the group consisting of cysteine, alanine and serine at position 354 (SEQ ID NO:5, wherein position 354 is selected from the group consisting of cysteine, alanine and serine); or
b) an amino acid sequence which is otherwise the one of a β-galactosidase variant having at least 95% identity with SEQ ID NO:2 (β-galactosidase variant as defined herein), but which comprises an amino acid residue selected from the group consisting of cysteine, alanine and serine at position 354. Non-limitative examples of β-galactosidase^{FS} are as defined in SEQ ID Nos 7, 10, 13, 16, 19, 22, 25 and 28, wherein position 354 is an amino acid residue selected from the group consisting of cysteine, alanine and serine.

In an embodiment, the lacZ^{FS} allele encodes a β-galactosidase^{FS} comprising:
a) an amino acid sequence which is otherwise as defined in SEQ ID NO:2, but which comprises a cysteine at position 354 (SEQ ID NO:4); in an embodiment, the lacZ^{FS} allele is as set forth in SEQ ID NO:3; or
b) an amino acid sequence which is otherwise the one of a β-galactosidase variant having at least 95% identity with SEQ ID NO:2 (β-galactosidase variant as defined herein), but which comprises a cysteine at position 354. Non-limitative examples of β-galactosidase^{FS} are as defined in SEQ ID NOs: 8, 11, 14, 17, 20, 23, 26 and 29.

In an embodiment, the lacZ^{FS} allele encodes a β-galactosidase^{FS} which is obtained from a β-galactosidase having a sequence as set forth in SEQ ID NO:2, by the substitution of the arginine by a cysteine at position 354 (R354C).

In an embodiment, the lacZ^{FS} allele encodes a β-galactosidase^{FS} which is obtained from a β-galactosidase variant having at least 95% identity with SEQ ID NO:2 (β-galactosidase variant as defined herein), by the substitution of the arginine by a cysteine at position 354 (R354C). In an embodiment, the lacZ^{FS} allele encodes a β-galactosidase^{FS} which is obtained from a β-galactosidase variant as set forth in SEQ ID NO: 6, 9, 12, 15, 18, 21, 24 or 27, by the substitution of the arginine by a cysteine at position 354 (R354C).

In a particular embodiment of any of these embodiments, the β-galactosidase^{FS} is 1026 amino acids in length.

### Amino Acid Numbering

In the present application, a specific numbering of amino acid residue positions is used for the characterization of the β-galactosidase. By alignment of the amino acid sequence of a β-galactosidase^{FS} protein or of a β-galactosidase variant, with the β-galactosidase protein defined in SEQ ID NO:2, it is possible to allot a number to an amino acid residue position in said β-galactosidase^{FS} or said β-galactosidase variant respectively, which corresponds with the amino acid residue position or numbering of the amino acid sequence shown in SEQ ID NO:2.

An alternative way of describing the amino acid numbering used in this application is to say that amino acid positions are identified by those ‘corresponding’ to a particular position in the amino acid sequence shown in SEQ ID NO:2. This is not to be interpreted as meaning the sequences of the present invention must include the amino acid sequence shown in SEQ ID NO:2. A skilled person will readily appreciate that β-galactosidase sequences vary among different bacterial strains. Reference to the amino acid sequence shown in SEQ ID NO:2 is used merely to enable identification of a particular amino acid location within any particular β-galactosidase. Such amino acid locations can be routinely identified using sequence alignment programs, the use of which are well known in the art.

### Polynucleotide of the Invention

In an aspect, the present invention provides a polynucleotide comprising or consisting of a lacZ^{FS} allele [encoding a β-galactosidase^{FS} of the invention. In an embodiment, the polynucleotide is a lacZ^{FS} allele [encoding a β-galactosidase^{FS}] of the invention. In an embodiment, the polynucleotide of the invention encodes a β-galactosidase^{FS} as defined herein. In an embodiment, the size of the polynucleotide of the invention is at least 3063 nucleotides, at least 3066 nucleotides, at least 3069 nucleotides, at least 3072 nucleotides, at least 3075 nucleotides, at least 3078 nucleotides or at least 3081 nucleotides. In an embodiment, the size of the polynucleotide of the invention is less than 5 kb or less than 4 kb. In an embodiment, the size of the polynucleotide ranges from a minimal size selected from the group consisting of at least 3063 nucleotides, at least 3066 nucleotides, at least 3069 nucleotides, at least 3072 nucleotides, at least 3075 nucleotides, at least 3078 nucleotides or at least 3081 nucleotides to a maximal size selected from the group consisting of 4 kb and 5 kb. In an embodiment, the size of the polynucleotide is 3078 or 3081 nucleotides.

In an embodiment, the polynucleotide of the invention consists of a lacZ^{FS} allele as defined herein, independently flanked on one side (in 5′ and in 3′) or on both sides of a nucleotide region ranging from 500 bp to 1 kb.

In an aspect, the present invention provides a polynucleotide comprising or consisting of a part of at least 100 nucleotides of the polynucleotide encoding a β-galactosidase^{FS} as defined herein, wherein said nucleotide part encompasses the codon corresponding to the residue 354 of said β-galactosidase^{FS}. The expression “codon corresponding to the residue 354 of said β-galactosidase^{FS}” means the codon 354 of the lacZ^{FS} allele as defined herein, wherein said codon corresponds to the residue 354 of the β-galactosidase^{FS}, wherein the amino acid sequence set forth in SEQ ID NO:2 is used for numbering. The position of the codon 354 of the lacZ^{FS} allele and the position of the residue 354 of the β-galactosidase^{FS} can easily be determined by the person skilled in the art, by aligning the part of at least 100 nucleotides or the β-galactosidase peptide coded by this part of at least 100 nucleotides with SEQ ID NO:1 or SEQ ID NO:2 respectively. In an embodiment, the polynucleotide comprises a part of the polynucleotide consisting of a lacZ^{FS} allele, wherein said nucleotide part encompasses the codon corresponding to the residue 354 of the encoded β-galactosidase^{FS}.

In an embodiment, the nucleotide part comprises or consists of at least 100 consecutive nucleotides of the polynucleotide comprising or consisting of a lacZ^{FS} allele as defined herein. In an embodiment, the nucleotide part comprises or consists of at least 200 consecutive nucleotides of the polynucleotide comprising or consisting of a lacZ^{FS} allele. In an embodiment, the nucleotide part comprises or consists of at least 300 consecutive nucleotides of the polynucleotide comprising or consisting of a lacZ^{FS} allele. In an embodiment, the nucleotide part comprises or consists of at least 400 consecutive nucleotides of the polynucleotide comprising or consisting of a lacZ^{FS} allele. In an embodiment, the nucleotide part comprises or consists of at least 500 consecutive nucleotides of the polynucleotide comprising or consisting of a lacZ^{FS} allele. In an embodiment, the nucleotide part comprises or consists of at least 1000 consecutive nucleotides of the polynucleotide comprising or consisting of a lacZ^{FS} allele. In an embodiment, the nucleotide part comprises or consists of at least 1500 consecutive nucleotides of the polynucleotide comprising or consisting of a lacZ^{FS} allele. In an embodiment, the nucleotide part comprises or consists of at least 2000 consecutive nucleotides of the polynucleotide comprising or consisting of a lacZ^{FS} allele.

In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, wherein the residue corresponding to residue 354 is not an arginine. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, wherein the residue corresponding to residue 354 is not an amino acid residue selected from the group consisting of arginine, histidine, glutamine and lysine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, wherein the residue corresponding to residue 354 is not an amino acid residue selected from the group consisting of arginine, histidine, glutamine, lysine, glutamic acid and asparagine. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, wherein the residue corresponding to the residue 354 is a cysteine or an equivalent amino acid thereof. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, wherein the residue corresponding to the residue 354 is a cysteine, alanine and serine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, wherein the residue corresponding to the residue 354 is a cysteine.

In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the sequence of which is at least 95% identical to, but different from, SEQ ID NO:2. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, the sequence of which is at least 95% identical to, but different from, SEQ ID NO:2 and wherein the residue corresponding to residue 354 is not an arginine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the sequence of which is at least 95% identical to, but different from, SEQ ID NO:2 and wherein the residue corresponding to residue 354 is not an amino acid residue selected from the group consisting of arginine, histidine, glutamine and lysine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the sequence of which is at least 95% identical to, but different from, SEQ ID NO:2 and wherein the residue corresponding to residue 354 is not an amino acid residue selected from the group consisting of arginine, histidine, glutamine, lysine, glutamic acid and asparagine. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, the sequence of which is at least 95% identical to, but different from, SEQ ID NO:2 and wherein the residue corresponding to the residue 354 is a cysteine or an equivalent amino acid thereof. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the sequence of which is at least 95% identical to, but different from, SEQ ID NO:2 and wherein the residue corresponding to the residue 354 is a cysteine, alanine and serine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the sequence of which is at least 95% identical to, but different from, SEQ ID NO:2 and wherein the residue corresponding to the residue 354 is a cysteine.

In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the amino acid sequence of which is a) an amino acid sequence which is otherwise as defined in SEQ ID NO:2, but which does not comprise an arginine at position 354 (SEQ ID NO:5, wherein position 354 is not an arginine); or b) an amino acid sequence which is otherwise the one of a β-galactosidase variant having at least 95% identity with SEQ ID NO:2 (β-galactosidase variant as defined herein), but which does not comprise an arginine at position 354; in an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS} as defined in SEQ ID Nos 7, 10, 13, 16, 19, 22, 25 or 28, wherein position 354 is not an arginine. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, the amino acid sequence of which is a) an amino acid sequence which is otherwise as defined in SEQ ID NO:2, but which does not comprise an amino acid residue selected from the group consisting of arginine, histidine, glutamine and lysine at position 354 (SEQ ID NO:5, wherein position 354 is not an amino acid residue selected from the group consisting of arginine, histidine, glutamine and lysine); or b) an amino acid sequence which is otherwise the one of a β-galactosidase variant having at least 95% identity with SEQ ID NO:2 (β-galactosidase variant as defined herein), but which does not comprise an amino acid residue selected from the group consisting of arginine, histidine, glutamine and lysine at position 354; in an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS} as defined in SEQ ID Nos 7, 10, 13, 16, 19, 22, 25 or 28, wherein position 354 is not an amino acid residue selected from the group consisting of arginine, histidine, glutamine and lysine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the amino acid sequence of which is a) an amino acid sequence which is otherwise as defined in SEQ ID NO:2, but which does not comprise an amino acid residue selected from the group consisting of arginine, histidine, glutamine, lysine, glutamic acid and asparagine at position 354 (SEQ ID NO:5, wherein position 354 is not an amino acid residue selected from the group consisting of arginine, histidine, glutamine, lysine, glutamic acid and asparagine); or b) an amino acid sequence which is otherwise the one of a β-galactosidase variant having at least 95% identity with SEQ ID NO:2 (β-galactosidase variant as defined herein), but which does not comprise an amino acid residue selected from the group consisting of arginine, histidine, glutamine, lysine, glutamic acid and asparagine at position 354; in an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS} as defined in SEQ ID Nos 7, 10, 13, 16, 19, 22, 25 or 28, wherein position 354 is not an amino acid residue selected from the group consisting of arginine, histidine, glutamine, lysine, glutamic acid and asparagine. In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS}, the amino acid sequence of which is a) an amino acid sequence which is otherwise as defined in SEQ ID NO:2, but which comprises a cysteine or an equivalent amino acid thereof at position 354 (SEQ ID NO:5, wherein position 354 is a cysteine or an equivalent amino acid thereof); or b) an amino acid sequence which is otherwise the one of a β-galactosidase variant having at least 95% identity with SEQ ID NO:2 (β-galactosidase variant as defined herein), but which comprises a cysteine or an equivalent amino acid thereof at position 354; in an embodiment, the nucleotide part, encompassing the codon corresponding to the residue 354 of said β-galactosidase^{FS} comprises or consists of a part of at least 100 nucleotides of the polynucleotide encoding a β-galactosidase^{FS} as defined in SEQ ID Nos 7, 10, 13, 16, 19, 22, 25 or 28, wherein position 354 is a cysteine or an equivalent amino acid thereof. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, the amino acid sequence of which is a) an amino acid sequence which is otherwise as defined in SEQ ID NO:2, but which comprises an amino acid residue selected from the group consisting of cysteine, alanine and serine at position 354 (SEQ ID NO:5, wherein position 354 is selected from the group consisting of cysteine, alanine and serine); or b) an amino acid sequence which is otherwise the one of a β-galactosidase variant having at least 95% identity with SEQ ID NO:2 (β-galactosidase variant as defined herein), but which comprises an amino acid residue selected from the group consisting of cysteine, alanine and serine at position 354; in an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS} as defined in SEQ ID Nos 7, 10, 13, 16, 19, 22, 25 or 28, wherein position 354 is an amino acid residue selected from the group consisting of cysteine, alanine and serine. In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS}, the amino acid sequence of which is a) an amino acid sequence which is otherwise as defined in SEQ ID NO:2, but which comprises a cysteine at position 354 (SEQ ID NO:4); or b) an amino acid sequence which is otherwise the one of a β-galactosidase variant having at least 95% identity with SEQ ID NO:2 (β-galactosidase variant as defined herein), but which comprises a cysteine at position 354; in an embodiment, the nucleotide part, encompassing the codon corresponding to the residue 354 of said β-galactosidase^{FS} comprises or consists of a part of at least 100 nucleotides of the polynucleotide encoding a β-galactosidase^{FS} as defined in SEQ ID NOs: 8, 11, 14, 17, 20, 23, 26 and 29.

In an embodiment, the nucleotide part, encompassing the codon corresponding to the residue 354 of said β-galactosidase^{FS}, comprises or consists of a part of at least 100 nucleotides of the polynucleotide encoding a β-galactosidase^{FS} which is obtained from a β-galactosidase having a sequence as set forth in SEQ ID NO:2, by the substitution of the arginine by a cysteine at position 354 (R354C). In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS} which is obtained from a β-galactosidase variant having at least 95% identity with SEQ ID NO:2 (β-galactosidase variant as defined herein), by the substitution of the arginine by a cysteine at position 354 (R354C). In an embodiment, the nucleotide part, encompassing the codon corresponding to the residue 354 of said β-galactosidase^{FS} comprises or consists of a part of at least 100 nucleotides of the polynucleotide encoding a β-galactosidase^{FS} which is obtained from a β-galactosidase variant as set forth in SEQ ID NO: 6, 9, 12, 15, 18, 21, 24 or 27, by the substitution of the arginine by a cysteine at position 354 (R354C).

Typically, the polynucleotide encompassed by the scope of the present invention is prepared using recombinant DNA techniques (i.e. recombinant DNA), as described herein. However, in an alternative embodiment of the invention, the polynucleotide could be synthesized, in whole or in part, using chemical methods well known in the art (see Caruthers M H et al., (1980) Nuc Acids Res Symp Ser 215-23 and Horn T et al., (1980) Nuc Acids Res Symp Ser 225-232).

A polynucleotide encoding a lacZ^{FS} protein as defined herein may be identified and/or isolated and/or purified from any lactic acid bacterium. Various methods are well known within the art for the identification and/or isolation and/or purification of polynucleotides.

By way of example, PCR amplification techniques to prepare more copies of a polynucleotide may be used once a suitable polynucleotide has been identified and/or isolated and/or purified.

By way of further example, a genomic DNA library may be constructed using chromosomal DNA from the lactic acid bacteria producing the β-galactosidase^{FS}. Based on the sequence of the β-galactosidase^{FS}, oligonucleotide probes may be synthesised and used to identify protein-encoding clones from the genomic library prepared from the lactic acid bacteria.

Alternatively, the polynucleotide of the invention may be prepared synthetically by established standard methods, e.g. the phosphoroamidite method described by Beucage S. L. et al., 1981, Tetrahedron Letters 22:1859-1869, or the method described by Matthes et al., 1984, EMBO J., 3:801-805. In the phosphoroamidite method, oligonucleotides are synthesised, e.g. in an automatic DNA synthesiser, purified, annealed, ligated and cloned in appropriate vectors.

The polynucleotide may be prepared by polymerase chain reaction (PCR) using specific primers, for instance as described in U.S. Pat. No. 4,683,202 or in Saiki R K et al., 1988, Science, 239:487-491.

The polynucleotide and the nucleic acids encompassed by the present invention may be isolated or substantially purified. By “isolated” or “substantially purified” is intended that the polynucleotides are substantially or essentially free from components normally found in association with the polynucleotide in its natural state. Such components include other cellular material, culture media from recombinant production, and various chemicals used in chemically synthesising the nucleic acids.

An “isolated” polynucleotide or nucleic acid is typically free of nucleic acid sequences that flank the nucleic acid of interest in the genomic DNA of the organism from which the nucleic acid was derived (such as coding sequences present at the 5′ or 3′ ends). However, the molecule may include some additional bases or moieties that do not deleteriously affect the basic characteristics of the composition.

### Vector

The invention is also directed to a vector comprising the polynucleotide of the invention. In an embodiment, this vector is a plasmid.

In an embodiment, the vector contains one or more selectable marker genes, such as a gene which confers antibiotic resistance e.g. ampicillin, kanamycin, chloramphenicol or tetracycline resistance. In an embodiment, the vector comprises a nucleotide sequence enabling the vector to replicate in the host cell in question. Examples of such sequences are the origins of replication of plasmids pUC19, pACYC177, pUBI 10, pE194, pAMBI and pIJ702.

A vector of the invention can be used to engineer a lactic acid bacterium of the invention.

Streptococcus thermophilus Strain Comprising a Polynucleotide of the Invention

The invention is directed to a Streptococcus thermophilus strain comprising a polynucleotide comprising or consisting of a lacZ^{FS} allele [encoding a β-galactosidase^{FS}] of the invention. In an embodiment, the Streptococcus thermophilus strain comprises a lacZ^{FS} allele [encoding a β-galactosidase^{FS}] of the invention.

For the avoidance of doubt, the Streptococcus thermophilus species is to be understood as a Streptococcus salivarius subsp. thermophilus strain.

In an embodiment, the Streptococcus thermophilus strain of the invention is a galactose-negative Streptococcus thermophilus strain. By the expression “galactose-negative”, it is meant a Streptococcus thermophilus strain which is not able to grow on galactose as a sole source of carbohydrate, in particular on a M17 medium supplemented with 2% galactose. In a particular embodiment, the “galactose-negative” phenotype is assayed by inoculating—into a M17 broth containing 2% galactose—an overnight culture of the S. thermophilus strain to be tested at 1% and incubating for 20 hours at 37° C., and wherein a pH of 6 or above at the end of incubation is indicative of a galactose-negative phenotype.

As described herein, “comprising a polynucleotide comprising or consisting of a lacZ^{FS} allele” or “comprising a lacZ^{FS} allele” means that the sole allele of the lacZ gene contained in the genome of the Streptococcus thermophilus strain is a lacZ^{FS} allele. In an embodiment, the Streptococcus thermophilus strain of the invention comprises, as the sole allele of its lacZ gene, a polynucleotide comprising or consisting of a lacZ^{FS} allele of the invention. It is not contemplated that the Streptococcus thermophilus strain of the invention comprises several alleles of the lacZ gene.

Such Streptococcus thermophilus strain may be engineered by:
a) replacing the allele of its lacZ gene by a polynucleotide comprising or consisting of a lacZ^{FS} allele of the invention; or
b) replacing a part of the allele of its lacZ gene by a corresponding polynucleotide comprising or consisting of a part of at least 100 nucleotides of the polynucleotide encoding a β-galactosidase^{FS} as defined herein, wherein said nucleotide part encompasses the codon corresponding to the residue 354 of said β-galactosidase^{FS}. By “corresponding polynucleotide”, it is meant the same portion of the lacZ allele encompassing the codon corresponding to the residue 354 of said β-galactosidase^{FS}

The replacement can be done using conventional techniques as defined herein.

In an embodiment, the Streptococcus thermophilus of the invention (comprising a lacZ^{FS} allele) is further characterized by its ability when tested by assay C, to lead to a slope of acidification between pH 6 and 5.3 of at least −0.005 UpH/min. In an embodiment, the slope of acidification between pH 6 and 5.3 is at least −0.006 UpH/min. In an embodiment, the slope of acidification between pH 6 and 5.3 is at least −0.007 UpH/min. In an embodiment, the slope of acidification between pH 6 and 5.3 is at least −0.008 UpH/min. In an embodiment, the slope of acidification between pH 6 and 5.3 is at least −0.009 UpH/min. In an embodiment, the slope of acidification between pH 6 and 5.3 is at least −0.01 UpH/min. In an embodiment, the slope of acidification between pH 6 and 5.3 is at least −0.02 UpH/min. In an embodiment, the slope of acidification between pH 6 and 5.3 is at least −0.03 UpH/min. In an embodiment, the slope of acidification between pH 6 and 5.3 is at least −0.04 UpH/min. In an embodiment, the slope of acidification between pH 6 and 5.3 is at least −0.05 UpH/min. In an embodiment, the slope of acidification between pH 6 and 5.3 is selected from the group of at least −0.005, −0.006, −0.007, −0.008, −0.009, −0.01, −0.02, −0.03, −0.04 and −0.05 UpH/min.

### Assay C (Acidification Kinetics in Milk)

UHT semi-skimmed milk “Le Petit Vendéen (“yoghurt milk”) containing 3% (w/v) milk powder (BBA, Lactalis), previously pasteurized 10 min at 90° C., is inoculated at 1% (v/v, about 10⁷ CFU/ml) with a culture of the S. thermophilus strain to be assayed (M17-carbohydrate-free resuspended cells from overnight culture grown in M17 supplemented with 3% sucrose). The inoculated milk flasks are statically incubated in a water bath at 43° C. (start of fermentation experiment) during 24 h, to obtain fermented milk. The acidifying properties of S. thermophilus strains were evaluated by recording the pH over time, during milk fermentation. The pH was monitored for 24 hours using the CINAC system (Alliance Instruments, France; pH electrode Mettler 405 DPAS SC, Toledo, Spain) as previously described. The pH was measured and recorded every 5 minutes. Using the CINAC v2.07 software, the following descriptors have been calculated:
the slope between pH 6.0 and pH 5.3 (UpH/minute) [Slope pH6-5.3];
the time corresponding to Vₘₐₓ (with Vₘₐₓ is the maximal velocity obtained during the fermentation experiment; T_{Vmax}), time (in minutes) calculated as from the start of fermentation experiment;
the pH_{STOP} corresponding to the pH value at V0, with V0 corresponding to a velocity which definitively becomes non-detectable, i.e., below 0.1 mupH/minutes (0.0001 UpH/min); by “definitively becomes”, it is meant that the velocity stays less than 0.1 mUpH/min for the remaining time of the assay C (i.e. up to 24 h at fermentation temperature); and
the time corresponding to the pH_{STOP} (TpH_{STOP}) [so, the time corresponding to V0, calculated as from the start of fermentation experiment].

In an embodiment, together with or independently from the slope of acidification determined by assay C, the Streptococcus thermophilus of the invention (comprising a lacZ^{FS} allele) is further characterized by its texturizing properties. Thus, the Streptococcus thermophilus of the invention can be characterized by the shear stress value it generates when use to obtain a fermented milk, as determined by assay D (i.e., at a shear rate of 350 s⁻¹).

In an embodiment, the shear stress value generated in a fermented milk obtained with a Streptococcus thermophilus of the invention, as determined by assay D, is at least 60, at least 120, at least 180 or at least 240 Pa. In an embodiment, the shear stress value generated in a fermented milk obtained with a Streptococcus thermophilus of the invention, as determined by assay D, is less than 60, less than 120, less than 180 or less than 240 Pa. In an embodiment, the shear stress value generated in a fermented milk obtained with a Streptococcus thermophilus of the invention, as determined by assay D, is both at least 60 or at least 120 and less than 180 or less than 240 Pa.

In an embodiment, the shear stress value generated in a fermented milk obtained with a Streptococcus thermophilus of the invention, as determined by assay D, is within a range selected from the group consisting of 0 to 59 Pa, 60 to 119 Pa, 120 to 179 Pa, 180 to 239 Pa and 240 to 300 Pa.

As a reference, the shear stress value generated in a fermented milk obtained with strain DGCC715 (DSM33036) was determined by assay D and was shown to be within the range 0-59 Pa. As another reference, the shear stress value generated in a fermented milk obtained with strain DGCC7710 (deposited as DSM28255) was determined by assay D and was shown to be within the range 120-179 Pa, more specifically to be about 150±15 Pa. The Streptococcus thermophilus DGCC7710 strain has been deposited by Danisco Deutschland GmbH under the Budapest Treaty at the Leibniz-Institut DSMZ-Deutsche Sammlung von Mikroorganismen und Zellkulturen, GmbH (Inhoffenstr. 7B, D-38124 Braunschweig), on Jan. 14, 2014 and have received the accession number DSM28255. We hereby confirm that the depositor, Danisco Deutschland GmbH (of Busch-Johannsen-Strasse 1, D-25899 Niebüll, Germany) has authorised the Applicant (DuPont Nutrition Biosciences ApS) to refer to the deposited biological material in this application. The applicant requests that a sample of the deposited microorganism stated herein may only be made available to an expert, until the date on which the patent is granted.

### Assay D

Strain inoculum preparation: 1.8 ml of a stock culture preserved at −80° C. is inoculated into 100 ml of a bulk starter medium in 250-ml flask and incubated for 18 h at 37° C. The bulk starter medium is obtained by adding into water 10% of high heat skimmed milk powder (BBA Lactalis), and agitating 30 minutes at room temperature; then, the medium is heat-treated 20 min at 120° C.

Milk preparation: 93% (w/w) of a commercial fresh milk [Candia, lait frais de montagne Grand Lait entier: 3.6% fat, 3.2% protein] and 7% (w/w) saccharose are mixed; the mixture is heat-treated at 90° C. for 10 min in water bath. Just before strain inoculation, 1 g/100 L (w/v) of sodium formiate is added.

Fermentation: the strain inoculum is added at 1% (v/v) into the milk and the inoculated milk is poured into 125 ml yogurt pot, and incubated at 43° C. until a pH of 4.6 is reached (pH is followed using a CINAC system; Alliance Instruments, France; pH electrode Mettler 405 DPAS SC, Toledo, Spain). Then, the fermented milk is slowly cooled in a well-ventilated cold incubator down to 6° C. The samples are stored for 7 days at 6° C.

Before shear stress determination, the samples are brought to 8° C. and stirred 5 times/5 s (1 turn=1 s) by using a spoon. A resting time of 5 min is applied (equilibration time) just before measurement. The shear stress of the sample is assessed using a rheometer (MCR Modular Compact Rheometer type 302, Anton Paar GmbH, Germany) equipped with the CC27 coaxial measuring system (Standard DIN 53019 and ISO 3219) and Peltier system C-PTD200-SN81154777. The viscometry test is done with a shear rate ramp varying from 0.1 s⁻¹ to 350 s⁻¹ in 31 points and from 350 s⁻¹ to 0.1 s⁻¹ in 31 points. The shear stress is continuously recorded. A logarithmic variable measuring point duration setting is used, with Up-curve initial value set at 10 s and final value set at 3 s, and Down-curve initial value set at 3 s and final value set at 10 s. The shear stress value at 350 s⁻¹ on the up-curve is selected to characterize the texturing properties of the S. thermophilus strain of the invention.

The inventors have shown that the Streptococcus thermophilus strains comprising a lacZ^{FS} allele of the invention can be used not only to ferment milk with an acceptable industrial time but also to have a fermented milk which does not undergo post acidification at fermentation temperature. The inventors have nicely shown that these Streptococcus thermophilus strains (comprising a lacZ^{FS} allele of the invention) can be defined by both the ratio LacS_{pH4.5} over LacZ_{pH4.5} as defined herein, and the ratio LacS_{pH}S over LacZ_{pH}S as defined herein in this strain. Indeed, the ratio LacS_{pH6} over LacZ_{pH6} represents the ability of the strain of the invention to utilize lactose and thus to acidify milk (lactic acid production) at the beginning of the manufacturing process down to the target pH, whereas the ratio LacS_{pH4.5} over LacZ_{pH4.5} represents the ability of this same strain to utilize lactose less efficiently and thus not to produce lactic acid when the target pH is reached. Thus, the inventors have shown that the formula (I) described herein can be used to characterize strains presenting an acidification kinetics in milk without post-acidification. In an embodiment, the Streptococcus thermophilus of the invention (comprising a lacZ^{FS} allele) is further characterized by a difference of efficiency of hydrolysis of the imported lactose (EH_{pH6}−EH_{pH4.5}) which is less than −0.5 calculated by the following formula (I):

in which formula (I), LacS_{pH6} and LacS_{pH4.5} represent the activity of lactose importation of the LacS permease calculated by assay A at pH 6 and at pH 4.5 respectively, and LacZ_{pH6} and LacZ_{pH4.5} represent the activity of lactose hydrolysis of the beta-galactosidase calculated by assay B at pH 6 and at pH 4.5 respectively.

Thus, a ΔEH as defined herein which is less than −0.5 means that the efficiency of hydrolysis of imported lactose at pH 4.5 (EH_{pH4.5}) [(i.e., importation of lactose into the bacteria by the LacS permease followed by the hydrolysis of the lactose by the beta-galactosidase)] is largely reduced as compared to the one at pH 6 (EH_{pH6}). In an embodiment, the Streptococcus thermophilus of the invention (comprising a lacZ^{FS} allele) is characterized by a ΔEH [as calculated by formula (I)] which is selected in the group consisting of less than −0.6, less than −0.7, less than −0.8, less than −0.9, less than −1, less than −1.1, less than −1.2, less than −1.3, less than −1.4 and less than −1.5.

In contrast, a ΔEH which is slightly positive, around 0 or slightly negative means that the efficiency of hydrolysis of imported lactose is as efficient in pH 4.5 as in pH 6. Such a ΔEH is characteristic of Streptococcus thermophilus strains which when used to ferment milk lead to a fermented milk undergoing post acidification.

It is also part of the invention that the Streptococcus thermophilus strain defined herein (comprising a lacZ^{FS} allele according to the invention) is further characterized by its ability to ferment milk with an acceptable industrial time followed by a fermented milk which does not undergo post acidification at fermentation temperature. This ability is defined herein as a “full STOP” phenotype and can be determined by the assay C as defined herein.

Thus, the full STOP phenotype is characterized by the fact that when the strain of the invention is inoculated to milk substrate and fermented according to assay C, the milk is fermented such that the pH of the fermented milk stops between 4 and 4.8 (pH_{STOP}), and the time between T_{Vmax} and TpH_{STOP} is less than 600 minutes. In an embodiment, the time between T_{Vmax} and TpH_{STOP} is less than 550 minutes. In an embodiment, the time between T_{Vmax} and TpH_{STOP} is less than 500 minutes.

In an embodiment, individually or in combination with the time between the Vmax and V0, the pH_{STOP} obtained using a strain of the invention by assay C is comprised between 4 and 4.6. In an embodiment, the pH_{STOP} obtained using a strain of the invention by assay C is comprised between 4 and 4.5. In an embodiment, the pH_{STOP} obtained using a strain of the invention by assay C is comprised between 4 and 4.4.

In an embodiment, the full STOP phenotype is characterized by the fact that when the strain of the invention is inoculated to milk substrate and fermented according to assay C, the milk is fermented such that the pH of the fermented milk stops between a range selected from the group consisting of between 4 and 4.8, between 4 and 4.6, between 4 and 4.5 and between 4 and 4.4, and the time between T_{Vmax} and TpH_{STOP} is selected from the group consisting of less than 600 minutes, less than 550 minutes and less than 500 minutes.

Thus, once the pH is stopped significantly quickly, the fermented dairy product can be kept at fermentation temperature for at least 24 hours, without the pH of the fermented product decreases (what gives high flexibility within the manufacturing process).

In a particular embodiment, the Streptococcus thermophilus strain of the invention as defined herein bears, as its lacZ gene, a lacZ^{FS} allele encoding a β-galactosidase^{FS} as defined in SEQ ID NO:4, in particular a lacZ^{FS} allele as defined in SEQ ID NO:3.

In a particular embodiment, the Streptococcus thermophilus strain of the invention as defined herein bears, as its lacZ gene, a lacZ^{FS} allele encoding a β-galactosidase^{FS} having at least 95% identity with SEQ ID NO:2, but which comprises a cysteine at position 354.

In a particular embodiment, the Streptococcus thermophilus strain of the invention as defined herein bears, as its lacZ gene, a lacZ^{FS} allele encoding a β-galactosidase^{FS}, the amino acid sequence of which is otherwise the one of a β-galactosidase variant having at least 95% identity with SEQ ID NO:2 (β-galactosidase variant as defined herein), but which comprises a cysteine at position 354. In a particular embodiment, the Streptococcus thermophilus strain of the invention bears, as its lacZ gene, a lacZ^{FS} allele encoding a β-galactosidase^{FS} as defined in SEQ ID NOs: 8, 11, 14, 17, 20, 23, 26 or 29.

In a particular embodiment, the invention is directed to a Streptococcus thermophilus strain corresponding to the Streptococcus thermophilus strain DGCC7984, the lacZ gene of which has been replaced by a lacZ^{FS} allele encoding a β-galactosidase^{FS} as defined in SEQ ID NO:4, in particular by a lacZ^{FS} allele as defined in SEQ ID NO:3. The Streptococcus thermophilus DGCC7984 strain has been deposited by Danisco Deutschland GmbH under the Budapest Treaty at the Leibniz-Institut DSMZ-Deutsche Sammlung von Mikroorganismen und Zellkulturen, GmbH (Inhoffenstr. 7B, D-38124 Braunschweig), on Jan. 14, 2014 and have received the accession number DSM28257. We hereby confirm that the depositor, Danisco Deutschland GmbH (of Busch-Johannsen-Strasse 1, D-25899 Niebüll, Germany) has authorised the Applicant (DuPont Nutrition Biosciences ApS) to refer to the deposited biological material in this application. The applicant requests that a sample of the deposited microorganism stated herein may only be made available to an expert, until the date on which the patent is granted. The expressions “DGCC7984 strain” is used interchangeably with the expression “DSM28257 strain”

### Use and Methods Based on the Polynucleotide or Vector of the Invention

In an embodiment, the invention is directed to the use of a polynucleotide or vector of the invention to obtain a Streptococcus thermophilus strain with a full STOP phenotype when used to ferment milk by assay C.

Thus, the polynucleotide or vector is used such that the resulting Streptococcus thermophilus strain comprises a lacZ^{FS} allele as the sole lacZ gene in its genome. In an embodiment, the polynucleotide or vector is used such that the allele of the lacZ gene or part thereof of the Streptococcus thermophilus strain is replaced by the polynucleotide of the invention; the replacement can be done using conventional techniques as defined herein.

In an aspect, the invention is directed to a method to prepare a Streptococcus thermophilus strain with a full STOP phenotype, comprising:
a) providing a Streptococcus thermophilus strain having a ratio of the activity of lactose importation of the LacS permease calculated by assay A at pH 4.5 over the activity of lactose hydrolysis of the beta-galactosidase calculated by assay B at pH 4.5 (LacS_{pH4.5} over LacZ_{pH4.5}) which is less than 5;
b) replacing the lacZ gene of said Streptococcus thermophilus strain with a polynucleotide (comprising or consisting of a lacZ^{FS} allele) of the invention; and
c) recovering the Streptococcus thermophilus strain(s) with a full STOP phenotype when used to ferment milk by assay C.

In an embodiment, step b) consists in replacing the lacZ gene of said Streptococcus thermophilus strain with a polynucleotide consisting of a lacZ^{FS} allele of the invention.

In an aspect, the invention is directed to a method to prepare a Streptococcus thermophilus strain with a full STOP phenotype, comprising:
a) providing a Streptococcus thermophilus strain having a ratio of the activity of lactose importation of the LacS permease calculated by assay A at pH 4.5 over the activity of lactose hydrolysis of the beta-galactosidase calculated by assay B at pH 4.5 (LacS_{pH4.5} over LacZ_{pH4.5}) which is less than 5;
b) replacing a part of the lacZ gene of said Streptococcus thermophilus strain by a corresponding polynucleotide comprising or consisting of a part of at least 100 nucleotides of the polynucleotide encoding a β-galactosidase^{FS} as defined herein, wherein said nucleotide part encompasses the codon corresponding to the residue 354 of said β-galactosidase^{FS}. By “corresponding polynucleotide”, it is meant the same portion of the lacZ allele encompassing the codon corresponding to the residue 354 of said β-galactosidase^{FS}; and
c) recovering the Streptococcus thermophilus strain(s) with a full STOP phenotype when used to ferment milk by assay C.

In an aspect, the invention is directed to a method to prepare a Streptococcus thermophilus strain with a full STOP phenotype, comprising:
a) providing a Streptococcus thermophilus strain having a ratio of the activity of lactose importation of the LacS permease calculated by assay A at pH 4.5 over the activity of lactose hydrolysis of the beta-galactosidase calculated by assay B at pH 4.5 (LacS_{pH4.5} over LacZ_{pH4.5}) which is less than 5;
b) modifying the lacZ gene of said Streptococcus thermophilus strain to have the same sequence as a lacZ^{FS} allele of the invention; and
c) recovering the lactic Streptococcus thermophilus strain(s) with a full STOP phenotype when used to ferment milk by assay C.

In an embodiment, any of the methods described herein to prepare a Streptococcus thermophilus strain with a full STOP phenotype is implemented on a medium containing lactose as the sole source of carbohydrate.

Within the use or methods of the invention, the ratio LacS_{pH4.5} over LacZ_{pH4.5} is determined as described herein. In an embodiment, the Streptococcus thermophilus strain of step a) has a ratio LacS_{pH4.5} over LacZ_{pH4.5} which is less than 5. In an embodiment, the Streptococcus thermophilus strain of step a) has a ratio LacS_{pH4.5} over LacZ_{pH4.5} which is less than 4. In an embodiment, the Streptococcus thermophilus strain of step a) has a ratio LacS_{pH4.5} over LacZ_{pH4.5} which is less than 3.

In an embodiment, the Streptococcus thermophilus strain of step a) is further characterized by its ability when tested by assay C, to lead to a slope of acidification between pH 6 and 5.3 of at least −0.005 UpH/min. In an embodiment, the slope of acidification between pH 6 and 5.3 is at least −0.006 UpH/min. In an embodiment, the slope of acidification between pH 6 and 5.3 is at least −0.007 UpH/min. In an embodiment, the slope of acidification between pH 6 and 5.3 is at least −0.008 UpH/min. In an embodiment, the slope of acidification between pH 6 and 5.3 is at least −0.009 UpH/min. In an embodiment, the slope of acidification between pH 6 and 5.3 is at least −0.01 UpH/min. In an embodiment, the slope of acidification between pH 6 and 5.3 is at least −0.02 UpH/min. In an embodiment, the slope of acidification between pH 6 and 5.3 is at least −0.03 UpH/min. In an embodiment, the slope of acidification between pH 6 and 5.3 is at least −0.04 UpH/min. In an embodiment, the slope of acidification between pH 6 and 5.3 is at least −0.05 UpH/min. In an embodiment, the Streptococcus thermophilus strain of step a) is further characterized by its ability when tested by assay C, to lead to slope of acidification between pH 6 and pH 4.5 which is selected from the group of at least −0.005, −0.006, −0.007, −0.008, −0.009, −0.01, −0.02, −0.03, −0.04 and −0.05 UpH/min.

In a further aspect, the invention is directed to a Streptococcus thermophilus strain obtained by the use or the method of the invention.

In a yet further aspect, the invention provides a Streptococcus thermophilus strain according to the invention produced by the method of the invention.

### Bacterial Composition

The invention is also directed to a bacterial composition comprising or consisting of at least one, preferably one, Streptococcus thermophilus strain of the invention. In one embodiment, the bacterial composition is a pure culture, i.e., comprises or consists of a single Streptococcus thermophilus strain of the invention. In another embodiment, the bacterial composition is a mixed culture, i.e. comprises or consists of the Streptococcus thermophilus strain(s) of the invention and at least one other microorganism, in particular at least one other bacterial strain. In one embodiment, the bacterial composition is a pure culture, i.e., comprises or consists of a single Streptococcus thermophilus strain of the invention. In another embodiment, the bacterial composition is a mixed culture, i.e. comprises or consists of the Streptococcus thermophilus strain(s) of the invention and at least one other bacterial strain. By “at least” one other bacteria strain, it is meant 1 or more, and in particular 1, 2, 3, 4 or 5 strains.

In an embodiment of any bacterial composition defined herein, either as a pure or mixed culture, the bacterial composition further comprises a food acceptable component, such as sugars (saccharose, trehalose), maltodextrin or minerals. In a particular embodiment, the bacterial composition defined herein does not comprise lactose.

In one embodiment, a bacterial composition of the invention comprises or consists of the Streptococcus thermophilus strain(s) of the invention, and one or more further lactic acid bacterium of the species selected from the group consisting of a Lactococcus species, a Streptococcus species, a Lactobacillus species including Lactobacillus acidophilus, an Enterococcus species, a Pediococcus species, a Leuconostoc species, a Bifidobacterium species and an Oenococcus species or any combination thereof. Lactococcus species include Lactococcus lactis, including Lactococcus lactis subsp. lactis, Lactococcus lactis subsp. cremoris and Lactococcus lactis subsp. lactis biovar diacetylactis. Bifidobacterium species includes Bifidobacterium animalis, in particular Bifidobacterium animalis subsp lactis. Other lactic acid bacteria species include Leuconostoc sp., Streptococcus thermophilus, Lactobacillus delbrueckii subsp. bulgaricus, and Lactobacillus helveticus.

In one embodiment, the bacterial composition comprises or consists of Streptococcus thermophilus strain(s) of the invention, and at least one Streptococcus thermophilus strain, different from the Streptococcus thermophilus strain(s) of the invention and/or at least one strain of the Lactobacillus species, and/or any combination thereof. In a particular embodiment, the bacterial composition comprises or consists of the Streptococcus thermophilus strain(s) of the invention, one or several strain(s) of the species Lactobacillus delbrueckii subsp. bulgaricus and/or one or several strain(s) of the species Lactobacillus helveticus and/or any combination thereof, and optionally at least one Streptococcus thermophilus strain, different from the Streptococcus thermophilus strain(s) of the invention. In a particular embodiment, the bacterial composition comprises or consists of the Streptococcus thermophilus strain(s) of the invention, at least one strain of species Streptococcus thermophilus, different from the Streptococcus thermophilus strain(s) of the invention, and a strain of the species Lactobacillus delbrueckii subsp. bulgaricus. In another particular embodiment, the bacterial composition comprises or consists of the Streptococcus thermophilus strain(s) of the invention, and a strain of the species Lactobacillus delbrueckii subsp. bulgaricus.

In one embodiment, the bacterial composition comprises or consists of the Streptococcus thermophilus strain(s) of the invention, a Lactococcus lactis subsp. lactis and/or a Lactococcus lactis subsp. cremoris.

In a particular embodiment of any bacterial composition defined herein, either as a pure or mixed culture, the bacterial composition further comprises at least one probiotic strain such as Bifidobacterium animalis subsp. lactis, Lactobacillus acidophilus, Lactobacillus paracasei, or Lactobacillus casei.

In a particular embodiment, the bacterial composition, either as a pure or mixed culture as defined above is in frozen, dried, freeze-dried, liquid or solid format, in the form of pellets or frozen pellets, or in a powder or dried powder. In a particular embodiment, the bacterial composition of the invention is in a frozen format or in the form of pellets or frozen pellets, in particular contained into one or more boxes or sachets. In another embodiment, the bacterial composition as defined herein is in a powder form, such as a dried or freeze-dried powder, in particular contained into one or more boxes or sachets.

In a particular embodiment, the bacterial composition of the invention, either as a pure culture or mixed culture as defined above, and whatever the format (frozen, dried, freeze-dried, liquid or solid format, in the form of pellets or frozen pellets, or in a powder or dried powder) comprises the Streptococcus thermophilus strain(s) of the invention in a concentration comprised in the range of 10⁵ to 10¹² cfu (colony forming units) per gram (cfu/g) of the bacterial composition. In a particular embodiment, the concentration of the Streptococcus thermophilus strain(s) within the bacterial composition of the invention is in the range of 10⁷ to 10¹² cfu per gram of the bacterial composition, and in particular at least 10⁷, at least 10⁸, at least 10⁹, at least 10¹⁰ or at least 10¹¹ cfu/g of the bacterial composition. In a particular embodiment, when in the form of frozen or dried concentrate, the concentration of the Streptococcus thermophilus strain(s) of the invention—as pure culture or as a mixed culture—within the bacterial composition is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

Manufacture of Product Using the Streptococcus thermophilus Strain of the Invention

In a further aspect, there is provided a method for manufacturing a fermented product comprising a) inoculating a substrate with the Streptococcus thermophilus strain or bacterial composition according to the invention and b) fermenting the inoculated substrate to obtain a fermented product. In a particular embodiment, the Streptococcus thermophilus strain(s) of the invention is inoculated as a bacterial composition as defined herein, such as a pure culture or a mixed culture. Preferably, the substrate is a milk substrate, more preferably milk. By “milk substrate”, it is meant milk of animal and/or plant origin. In a particular embodiment, the milk substrate is of animal origin, in particular of any mammals, such as cow, goat, sheep, buffalo, zebra, horse, donkey, or camel, and the like. The milk may be in the native state, a reconstituted milk, a skimmed milk, or a milk supplemented with compounds necessary for the growth of the bacteria or for the subsequent processing of fermented milk. Preferably, the milk substrate comprises solid items. Preferably, the solid items comprise or consist of fruits, chocolate products, or cereals. Preferably, the fermented product is a fermented dairy product.

The present invention also provides in a further aspect the use of the Streptococcus thermophilus strain or bacterial composition according to the present invention to manufacture a food or feed product, preferably a fermented dairy product.

The invention is also directed to a fermented dairy product, which is obtained using the lactic acid bacteria strain(s) or bacterial composition of the invention, in particular obtained or obtainable by the method of the invention. Thus, the invention is directed to a fermented dairy product comprising the Streptococcus thermophilus strain(s) of the invention. In a particular embodiment, the fermented dairy food product of the invention is fresh fermented milk.

The Streptococcus thermophilus strain or bacterial composition according to the invention finds an advantageous use in various dairy applications (as particular embodiments of a method for manufacturing a fermented product described herein).

In an aspect, the Streptococcus thermophilus strain or bacterial composition according to the invention finds use in the manufacture of stirred yoghurt. The manufacture of stirred yogurt comprises fermenting a milk substrate previously inoculated with the Streptococcus thermophilus strain or bacterial composition according to the invention, optionally storing the stirred yoghurt in a storage tank, and finally packing the stirred yoghurt into packages. This process involves cooling the stirred yoghurt between the end of the fermentation (i.e., once the target pH has been reached) and the packing step in order to stop further acidification of the stirred yoghurt, such that the stirred yoghurt is packed at a temperature between 15 and 22° C. Because this cooling step is time- and resource- (energy) consuming, yoghurt manufacturers look for packing the stirred yoghurt at a higher temperature; packing at a higher temperature also has the advantage of improving the texture of the stirred yoghurt in the packages (see example 8); however, packing at a higher temperature is not acceptable for yoghurt manufacturers with the bacterial compositions currently on the market, since the stirred yoghurt has been shown to be too acidic. The Streptococcus thermophilus strain or bacterial composition according to the invention solves this issue, enabling the yoghurt manufacturers to pack the stirred yoghurt at a higher temperature while obtaining a product with an acceptable pH. This can be achieved by either cooling the stirred yoghurt at a temperature higher than 22° C. or by bypassing the cooling step. Thus, the invention is also directed to the use of the Streptococcus thermophilus strain or bacterial composition according to the invention in the manufacture of stirred yoghurt. In a particular embodiment, the invention is also directed to the use of the Streptococcus thermophilus strain or bacterial composition according to the invention in the manufacture of stirred yoghurt, wherein the packing step of the stirred yoghurt is carried out at a temperature which is at least 23° C. The invention is also directed to a process to manufacture stirred yoghurt comprising (a) fermenting a milk substrate, in particular milk, inoculated with the Streptococcus thermophilus strain or bacterial composition according to the invention, to obtain a stirred yoghurt (with a pH from 4.2 to 4.7, more preferably from 4.45 to 4.6), (b) cooling the stirred yoghurt and (c) packing the stirred yoghurt, wherein the temperature of cooling and packing is at least 23° C. (the temperature of cooling and packing being one temperature). By “at least 23° C.” in the context of the temperature of cooling and packing, it is meant at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C. and at least 40° C. In a particular embodiment, the temperature of cooling and packing is equals to or less than the fermentation temperature (i.e., typically less than 43° C.). In a particular embodiment, the cooling and packing temperature is at least 23° C. and equals to or less than 43° C. As shown in example 8, packing at a temperature of 35° C. gives a pH over time similar to the one of a stirred yoghurt packed at 20° C., while at the same time improving the texture of the stirred yoghurt. The invention is also directed to a process to manufacture stirred yoghurt comprising (a) fermenting a milk substrate, in particular milk, with the Streptococcus thermophilus strain or bacterial composition according to the invention, to obtain a stirred yoghurt (with a pH from 4.2 to 4.7, more preferably from 4.45 to 4.6), and (b) packing this stirred yoghurt, wherein the process does not comprise any cooling step between end of fermentation and packing. In this embodiment, the temperature of cooling and packing is equal to the fermentation temperature (i.e., typically 42-43° C.). In an embodiment, the process to manufacture stirred yoghurt as described herein further comprises transferring the packages into a storage cold room (i.e., less than 8° C.).

In another aspect, the Streptococcus thermophilus strain or bacterial composition according to the invention finds use in the manufacture of set yoghurt. The manufacture of set yogurt involves cooling the packages containing the set yoghurt once the desired pH is obtained (with a pH from 4.2 to 4.7, more preferably from 4.45 to 4.6; considered as the end of the fermentation), to stop further acidification of the product. This cooling step is carried out in a cooling room (also called cooling chamber or cooling tunnel), before transfer of the packages into a storage cold room (i.e., less than 8° C.). With conventional starter cultures, it is important to stop further growth quickly after fermentation, which means that a temperature of about 35° C. should be reached within 30 minutes after end of fermentation, and 18-20° C. after another 30-40 minutes. Typically, the total cooling time is about 65-70 minutes for small packages and about 80-90 minutes for large packages. Because this cooling step is time- and resource- (energy) consuming, yoghurt manufacturers look for reducing the time spent in the cooling room; however, reducing this time is not acceptable for yoghurt manufacturers with the bacterial compositions currently on the market, since the yoghurt products have been shown to be too acidic. The Streptococcus thermophilus strain or bacterial composition according to the invention solves this issue, by enabling the yoghurt manufacturers to play with the period of time to reach a temperature of 18-20° C., while obtaining a product with an acceptable pH. In a particular embodiment, the invention is directed to the use of the Streptococcus thermophilus strain or bacterial composition according to the invention in the manufacture of set yoghurt, wherein the time needed for a set yoghurt contained in a package to reach a temperature of 18-20° C. (starting from the end of the fermentation) is increased as compared to a time of 65-70 minutes for small packages (herein defined as a size from 0.1 to 0.2 kg) and a time of 80-90 minutes for large packages (herein defined as a size from 0.4 to 0.6 kg). In a particular embodiment, the time needed for a set yoghurt contained in a package to reach a temperature of 18-20° C. is at least 100 minutes, at least 120 minutes, at least 180 minutes or at least 240 minutes. This can be achieved by several ways giving high flexibility to the dairy manufacturers, e.g., by bypassing the cooling step (i.e., bypassing the step in the cooling room) or by delaying the time between the end of fermentation and the time of entry into the cooling room. The invention is directed to a process to manufacture set yoghurt comprising a) packing a milk substrate, in particular milk, inoculated with the Streptococcus thermophilus strain or bacterial composition according to the invention into packages, (b) fermenting the inoculated milk substrate (contained in the packages) to obtain a set yoghurt (with a pH from 4.2 to 4.7, more preferably from 4.45 to 4.6), and c) handling the packages such that the time needed for the set yoghurt in the packages to reach a temperature of 18-20° C. is at least 100 minutes, at least 120 minutes, at least 180 minutes or at least 240 minutes. In a particular embodiment, the process to manufacture set yoghurt as described herein further comprises d) transferring the packages into a storage cold room (i.e., less than 8° C.). In an embodiment, the invention is directed to a process to manufacture set yoghurt comprising a) packing a milk substrate, in particular milk, inoculated with the Streptococcus thermophilus strain or bacterial composition according to the invention into packages, and b) fermenting the inoculated milk substrate to obtain a set yoghurt (with a pH from 4.2 to 4.7, more preferably from 4.45 to 4.6), wherein said process does not comprise a cooling step in a cooling room. In a particular embodiment, the process to manufacture set yoghurt as described herein further comprises c) transferring the packages into a storage cold room (i.e., less than 8° C.). In an embodiment, the invention is directed to a process to manufacture set yoghurt comprising a) packing a milk substrate, in particular milk, inoculated with the Streptococcus thermophilus strain or bacterial composition according to the invention into packages, b) fermenting the inoculated milk substrate to obtain a set yoghurt (with a pH from 4.2 to 4.7, more preferably from 4.45 to 4.6), c) keeping the set yoghurt in the packages at room temperature (i.e., higher than 20° C.) for at least 30 minutes, at least 45 minutes or at least 60 minutes after the end of fermentation; and d) incubating the packages in a cooling chamber in order the set yoghurt contained in the package reaches a temperature of 18-20° C.

In another aspect, the Streptococcus thermophilus strain or bacterial composition according to the invention finds use in the storage of fermented milk, such as stirred yoghurt and set yoghurt. At the end of the process of manufacture (including the packing and cooling), the fermented milks are stored in storage cold room at a temperature which is typically less than 8° C., until distribution. As shown in example 9, a yoghurt manufactured with a strain of the invention stored at 10° C. keeps a stable pH until 45 days (by stable, it is meant a variation of pH with is less than 0.1 unit). Thus, the invention is also directed to a process to manufacture and store a fermented milk, comprising a) fermenting a milk substrate, in particular milk, with the Streptococcus thermophilus strain or bacterial composition according to the invention, to obtain a fermented milk (with a pH from 4.2 to 4.7, more preferably from 4.45 to 4.6), b) optionally cooling the fermented milk to a temperature of 18-20° C., and c) storing the packages containing the fermented milk, the packing step occurring either before or after the fermentation step, but before the optional cooling step, wherein the storage is carried out at a temperature higher than 8° C.; in an embodiment, the storage is carried out at a temperature equals to or higher than 10° C., and optionally less than 20° C., preferably less than 15° C. In a particular embodiment, the time of storage at a temperature higher than 8° C. (preferably at a temperature equals to or higher than 10° C., and optionally less than 20° C., preferably less than 15° C.) is less than 24 hours.

### Product

Any product, which is prepared from, contains or comprises a Streptococcus thermophilus strain or bacterial composition of the invention is contemplated in accordance with the present invention.

Suitable products include, but are not limited to a food or a feed product.

These include, but are not limited to, fruits, legumes, fodder crops and vegetables including derived products, grain and grain-derived products, dairy foods and dairy food-derived products, meat, poultry and seafood. Preferably, the food or feed product is a dairy, meat or cereal product.

The term “food” is used in a broad sense and includes feeds, foodstuffs, food ingredients, food supplements, and functional foods. Here, the term “food” is used in a broad sense—and covers food for humans as well as food for animals (i.e., a feed). In a preferred aspect, the food is for human consumption.

As used herein the term “food ingredient” includes a formulation, which is or can be added to foods and includes formulations which can be used at low levels in a wide variety of products that require, for example, acidification or emulsification.

As used herein, the term “functional food” means a food which is capable of providing not only a nutritional effect and/or a taste satisfaction, but is also capable of delivering a further beneficial effect to consumers. Although there is no legal definition of a functional food, most of the parties with an interest in this area agree that there are foods marketed as having specific health effects.

The Streptococcus thermophilus strain of the present invention may be—or may be added to—a food ingredient, a food supplement, or a functional food.

The food may be in the form of a solution or as a solid—depending on the use and/or the mode of application and/or the mode of administration.

The Streptococcus thermophilus strain of the present invention can be used in the preparation of food products such as confectionery products, dairy products, meat products, poultry products, fish products or bakery products.

By way of example, the Streptococcus thermophilus strain can be used as an ingredient to prepare soft drinks, a fruit juice or a beverage comprising whey protein, teas, cocoa drinks, milk drinks and lactic acid bacteria drinks, yoghurt, drinking yoghurt and wine.

Preferably a food as described herein is a dairy product. More preferably, a dairy product as described herein is one or more of the following: a yoghurt, a cheese (such as an acid curd cheese, a hard cheese, a semi-hard cheese, a cottage cheese), a buttermilk, a quark, a sour cream, kefir, a fermented whey-based beverage, a koumiss, a milk beverage, a yoghurt drink, a fermented milk, a matured cream, a cheese, a fromage frais, a milk, a dairy product retentate, a process cheese, a cream dessert, or an infant milk.

Preferably, a food as described herein is a fermented food product. More preferably, a food as described herein is a fermented dairy product—such as a fermented milk, a yoghurt, a cream, a matured cream, a cheese, a fromage frais, a milk beverage, a processed cheese, a cream dessert, a cottage cheese, a yoghurt drink, a dairy product retentate, or an infant milk.

Preferably the dairy product according to the invention comprises milk of animal and/or plant origin.

Milk is understood to mean that of animal origin, in particular of any mammals such as cow, goat, sheep, buffalo, zebra, horse, donkey, or camel, and the like. The term milk also applies to what is commonly called vegetable milk, that is to say extracts of plant material which have been treated or otherwise, such as leguminous plants (soya bean, chick pea, lentil and the like) or oilseeds (colza, soya bean, sesame, cotton and the like), which extract contains proteins in solution or in colloidal suspension, which are coagulable by chemical action, by acid fermentation and/or by heat. Finally, the word milk also denotes mixtures of animal milks and of vegetable milks.

In one embodiment, the term “milk” means commercial UHT milk supplemented with 3% (w/w) of semi-skimmed milk powder pasteurized by heating during 10 min+/−1 min. at 90° C.+/−0.2° C.

In the field of dairy applications, the use of a fermented milk, such as a yoghurt, manufactured with the Streptococcus thermophilus strain or bacterial composition according to the invention is advantageous when mixed with warm flavors (such as coffee or chocolate flavors); indeed, not only the high pH of the yoghurt obtained with the strain of the invention but also the stability of this pH (no post-acidification) suppress the acidic perception in the final product and improves its mildness; these advantages render the use of warm flavors, like coffee or chocolate flavors, compatible with flavored-yoghurt manufacture. In another embodiment, the Streptococcus thermophilus strain or bacterial composition according to the invention is advantageous when used for the manufacture of Ryazhenka-type products (eastern Europe), also called “Brown-yogurts” (Asian countries) (fermentation of over-cooked milks developing caramel aromatic notes); indeed, conventional starter cultures developing yoghurt acidic note are not compatible with this type of fermented milk products.

### Percentage of Identity of a β-Galactosidase

A percentage of identity of at least 95% to SEQ ID NO:2 means a percentage of identity selected from the group consisting of at least 95%, at least 96%, at least 97%, at least 98% and at least 99%.

In an embodiment, though the sequence of the β-galactosidase is different from SEQ ID NO:2, the size of the β-galactosidase variant is the same as the β-galactosidase as defined in SEQ ID NO:2 (1026 amino acid residues).

Comparisons of sequences can be conducted by eye, or more usually, with the aid of readily available sequence comparison programs. These commercially or freely available computer programs can calculate similarity or identity values between two or more sequences.

A percentage of identity may be calculated over aligned, contiguous sequences, i.e. one sequence is aligned with regards to another sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the downstream amino acid residues to be put out of alignment, thus potentially resulting in a large reduction of the identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity. These more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap (gap extension penalty). This is the most commonly used gap scoring system. High gap penalties will of course produce optimised alignments with fewer gaps. Most alignment programs allow the gap penalties to be modified. However, it is possible to use the default values when using such software for sequence comparisons, because these default values have been adjusted to provide relevant results in most cases. Calculation of the maximum percentage of identity therefore firstly requires the production of an optimal alignment, taking into consideration gap penalties. A suitable computer program for carrying out such an alignment is Vector NTI (Invitrogen Corp.). An example of software that can perform sequence comparisons includes, but is not limited to, the BLAST package (see Ausubel et al., 1999, Short Protocols in Molecular Biology, 4th Ed—Chapter 18).

Although the alignment quality can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom comparison table if supplied (see user manual for further details). Alternatively, percentage of similarity may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate a percentage of sequence similarity, preferably a percentage of sequence identity. The software typically does this as part of the sequence comparison and generates a numerical result.

In an embodiment, the degree of identity with regards to a protein (amino acid) sequence is determined over at least 50 contiguous amino acids, at least 100 contiguous amino acids, at least 150 contiguous amino acids, at least 200 contiguous amino acids or at least 250 contiguous amino acids.

In an embodiment, the degree of identity with regards to an amino acid or protein sequence may be determined over the whole sequence of SEQ ID NO:2.

In an embodiment, the sequences [sequence of the β-galactosidase to be compared and SEQ ID NO:2] are aligned by a global alignment program and the sequence identity is calculated by identifying the number of exact matches identified by the program divided by the length of the sequence of the β-galactosidase to be compared.

In an embodiment, the degree of sequence identity between the sequence of the β-galactosidase to be compared and SEQ ID NO:2 is determined by: 1) aligning the two sequences by any suitable alignment program using the default scoring matrix and default gap penalties, 2) identifying the number of exact matches, where an exact match is where the alignment program has identified an identical amino acid in the two aligned sequences on a given position in the alignment and 3) dividing the number of exact matches with the length of the sequence of the β-galactosidase to be compared.

In an embodiment, the global alignment program is selected from the group consisting of CLUSTAL and BLAST, in particular CLUSTAL, using the default parameters, and the sequence identity is calculated by identifying the number of exact matches identified by the program divided by the length of the subject sequence.

In an embodiment, the global alignment program is CLUSTAL using the default parameters, and the sequence identity is determined with the BioEdit software (http://www.mbio.ncsu.edu/BioEdit/bioedit.html) [selecting the “Sequence” drop-down menu, then selecting the “Pairwise alignment” sub-menu, then selecting the “Calculate identity/similarity for two sequences” menu item].

### General Recombinant DNA Methodology Techniques

The present invention employs, unless otherwise indicated, conventional techniques of biochemistry, molecular biology, microbiology and recombinant DNA, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention will now be further described by way of Examples, which are meant to serve to assist one of ordinary skill in the art in carrying out the invention and are not intended in any way to limit the scope of the invention.

### MATERIAL AND METHODS

### Strains and Growth Conditions

The S. thermophilus strains (ST) disclosed in the present application were grown at 37° C. in M17 broth (Oxoid, supplier reference CM0817) supplemented with 30 g/L of lactose and if necessary, with addition of 15 g/L Agar Bacteriologic Type A (Biokar, supplier reference #A1010HA), or at 43° C. in milk (UHT semi-skimmed milk “Le Petit Vendéen”+3% milk powder BBA Lactalis). Autoclaved M17 broth was supplemented with 0.2 μm filtered lactose, sucrose, galactose or glucose. Frozen stocks of ST strains were obtained by half-diluting in M17 with 50% glycerol an overnight culture grown in M17 broth supplemented with 30 g/L sucrose, and stored at −20° C.

Transfer of the lacZ Allele of the DGCC12456 Strain into the Genome of 2 Other S. thermophilus Strains

A 1198-bp PCR product bearing the lacZ gene of the DGCC12456 strain was obtained using primers lacZ_F5 (5′-GTAACTTCGTAGGATACAGTG-3′) and lacZ_R6 (5′-CAGAGTTACCCATTGTGTGC-3′). The PCR product was then purified using QIAquick PCR Purification Kit (Qiagen) and eluted in DNase free water. The concentration of the PCR product was determined using NanoDrop 2000 spectrophotometer (Thermo Scientific, Wilmington, MA). The size and the purity of the PCR product were verified by gel-based capillary electrophoresis QIAxcel® system (Qiagen, Hilden, Germany). Strains DGCC715 and DGCC11231 were transformed with the 1198-bp PCR product by natural competence accordingly to Dandoy et al. (2011). Mutants having their lacZ gene replaced by the lacZ allele of the DGCC12456 strain were selected (the presence of the lacZ allele of the DGCC12456 strain was checked by sequencing).

Verification by Sequencing of the Presence of the lacZ Allele of the DGCC12456

PCR amplification of the β-galactosidase gene was performed using primers lacS_F1 (5′ GTAACTTCGTAGGATACAGTG-3′) and lacZ_R7 (5′-CAGAGTTACCCATTGTGTGC-3′), [incubation step at 98° C., 5 min, followed by 33 cycles of 98° C., 45 s; 58° C., 30 s; 68° C., 3 min, with a final extension step at 72° C., 7 min]. The PCR product of 1198-bp was then treated with Illustra™ ExoProStar™ according to the manufacturer's instructions (GE Healthcare). Sequencing reactions were performed by using the BigDye® Terminator v3.1 Cycle Sequencing kit (Life Technologies) according to the manufacturer's instructions using an AB3500 (Applied Biosystems™), and primers listed in Table 1.

TABLE 1
list of primers used for amplification
and sequencing of the fragment of lacZ
used for transformation

| Primers | Sequence 5′ - - - 3′ | SEQ ID |
|---|---|---|
| lacS_F1 | CTTGACTGCAGCTGAACTC | SEQ ID NO 32 |
| lacZ_R7 | CTCGACTACAAAGTTAACTGG | SEQ ID NO 33 |
| lacZ_R6 | CAGAGTTACCCATTGTGTGC | SEQ ID NO 34 |
| qLacZ_R4 | AGGTTGGCTTCATCGATAAC | SEQ ID NO 35 |
| qLacZ_F1 | CATCACCTTCTGTAACGATGC | SEQ ID NO 36 |
| LacZ_F5 | GTAACTTCGTAGGATACAGTG | SEQ ID NO 37 |
| qLacZ_F3 | AGGACGTTGTATCACTGAAG | SEQ ID NO 38 |

### LacS Activity [Assay A]

Streptococcus thermophilus strains were grown on M17 media containing 30 g/L of sucrose as sole carbon source overnight at 37° C. When cells reached the stationary phase, they were transferred (at 0.05 uDO/mL) in 1 volume of M17 media containing 30 g/L of lactose as sole carbon source and they were incubated for 2 hours at 42° C. Strain cultures were centrifuged at room temperature (3500 g), the supernatant was removed and cells were resuspended in 0.5 volume of 4% (w/v) glycerophosphate. This washing step was applied twice. 1.8 mL of cell suspension in 4% glycerophosphate were incubated for 2 minutes at 42° C. Then, 0.2 mL of lactose solution (70 g/L of lactose+0.1 M potassium phosphate buffer) was added [the lactose solution pH was previously adjusted at pH 4.5 or at pH 6, depending on the measurement needed]. The mix was incubated for 3 additional minutes at 42° C. The reaction was blocked by filtrating on 0.22 μm filter in order to remove cells. Then, the lactose in the filtrated solution was assayed on an HPLC using the following protocol. The solution was diluted 10-fold in water and 10 μL were injected on an Agilent 1200 HPLC (high-performance-liquid-chromatography). The elution was done in isocratic mode with pure water at 0.6 mL/min. Molecules were separated in 40 min onto a Pb²⁺ ion exchange column (SP-0810 Shodex® 300 mm×8 mm×7 μm) column. Sugars were detected with refractometer. Quantification was performed by external calibration.

The activity of lactose importation of the LacS permease is calculated as follows:

$$LacS activity = ( {[ lactose ]}_{initial} - {[ lactose ]}_{3 min} ) / ( DO \times time ) , expressed in μ mol / ( uDO . min ) ,$$

wherein:
[lactose]ᵢₙᵢₜᵢₐₗ is the initial concentration in μmol/mL
[lactose]₃ₘᵢₙ is the concentration in μmol/mL after 3 minutes at 42° C.
DO is the bacterial density in uDO/mL
time is the experiment duration in minutes (in the present case, 3 minutes).

### LacZ Activity [Assay B]

A fresh overnight culture of the Streptococcus thermophilus strain to be assayed in M17 containing 30 g/L lactose was obtained and used to inoculate at 1% (v/v) 10 ml of fresh M17 containing 30 g/L lactose. Cells were harvested by centrifugation (6000 g, 10 min, 4° C.) after 3 hours of growth on M17 containing 30 g/L lactose at 42° C., washed in 1.5 ml of cold lysis buffer (KPO4 0.1 M), and resuspended in 300 μl of cold lysis buffer. EDTA-free protease inhibitors “cOmplete™” (Roche, supplier reference 04693132001) was added to the lysis buffer as described by the supplier. Cells were disrupted by the addition of 100 mg glass beads (150-212 μm, Sigma G1145) to 250 μl of resuspended cells and oscillation at a frequency of 30 cycles/s for 6 min in a MM200 oscillating mill (Retsch, Haan, Germany). Cell debris and glass beads were removed by centrifugation (14000 g, 15 min, 4° C.), and the supernatant was transferred into a clean 1.5 mL centrifuge tube kept on ice. Total protein content was determined by using the FLUKA Protein Quantification Kit-Rapid (ref 51254). The beta-galactosidase activity in the cell extracts was determined spectrophotometrically by a monitoring of the hydrolysis of O-nitro-Phenol-Beta-Galactoside (ONPG) into galactose and O-nitro-phenol (ONP). Twenty μL of the cell extract were mixed with 135 μL of React Buffer (NaPO₄ 100 mM; KCl 10 mM; MgSO₄ 1 mM; ONPG 3 mM+Beta Mercapto Ethanol 60 mM, pH=6). The production of ONP leads to a yellow color into the tube. When the yellow color was appearing, the reaction was blocked by adding 250 μL of Stopping buffer (Na₂CO₃ 1 M). The optical density at 420 nm was recorded using a Synergy HT multi-detection microplate reader (BIO-TEK). One unit of beta-galactosidase corresponds to the amount of enzyme that catalyzes the production of 1 μmole ONP per minute under the assay conditions. Beta-galactosidase activity was calculated as follows:

LacZ activity=dOD×V/[dt×l×ε×Qprot], expressed in mol/(mg of total protein extract·min), wherein:
dOD is the variation of optical density (OD) at 420 nm between the blank and the tested sample
V is the volume of the reaction in which the optical density is measured (herein 250 μL)
dt=represent the duration in minutes between the addition of the 20 μL of bacterial extract and the addition of the 250 μL stopping buffer
l=optical path length (herein 0.73 cm)
ε=molar attenuation coefficient of ONP (herein 4500 cm²/μmol)
Qprot=quantity of protein in the cuvette (in mg)

### Milk Acidifying Performance [Assay C]

The acidifying properties of S. thermophilus strains were evaluated by recording the pH over time, during milk fermentation as follow: UHT semi-skimmed milk “Le Petit Vendéen (“yoghurt milk”) containing 3% (w/v) milk powder (BBA, Lactalis), previously pasteurized 10 min at 90° C., was inoculated at 1% (v/v, about 10⁷ CFU/ml) with a culture of the S. thermophilus strain to be assayed (M17-carbohydrate-free resuspended cells from overnight culture grown in M17 supplemented 3% sucrose). The inoculated milk flasks were statically incubated in a water bath at 43° C. during 24 h. The pH was monitored during the incubation using the CINAC system (Alliance Instruments, France; pH electrode Mettler 405 DPAS SC, Toledo, Spain) as previously described. The pH was measured and recorded every 5 minutes.

### Results

### Example 1: Isolation of a Streptococcus thermophilus Displaying a Full-Stop Phenotype

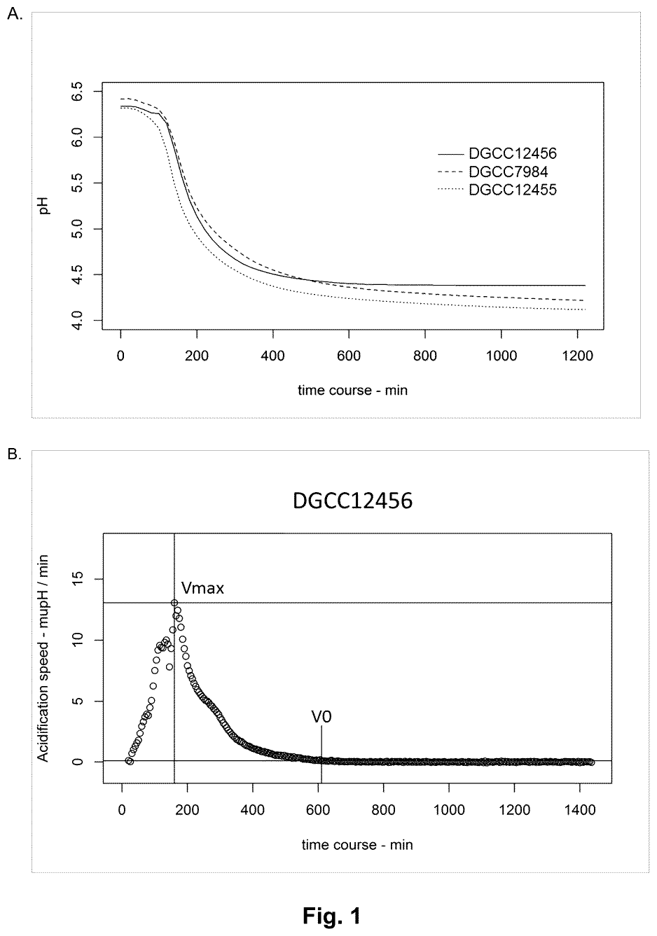

Dilutions of a culture of the DGCC7984 strain were plated onto the surface of M17 supplemented with 5 g/L sucrose agar plates. Upon incubation for 48 hours at 37° C., 2 isolated colonies of the DGCC7984 strain were picked and propagated for 24 hours in M17 broth supplemented with 20 g/L sucrose at 37° C. These two subclones of DGCC7984 strain were named DGCC12455 and DGCC12456. Acidification properties of strain DGCC12455 and DGCC12456 were investigated as follow: the 2 strains were inoculated into M17 broth supplemented with lactose 30 g/L and then incubated at 37° C. overnight. The cultures were washed (v/v) in tryptone-salt solution (tryptone 1 g/L, NaCl 8.5 g/L) as follow: the cultures were centrifugated at 4000 rpm for 5 minutes; the pellets were resuspended in 10 mL of tryptone-salt solution. The washed cultures were inoculated at 1% (v/v) into 100 mL of UHT half-skimmed milk containing 3% (w/v) of milk powder and pasteurized at 90° C. for 10 minutes. The flasks were incubated in a water bath at 43° C. and the pH was measured and recorded online using a CINAC system (FIG. 1A). The slope between pH 6.0 and pH 5.3 (−UpH/minute), representing the velocity between pH 6 and pH 5.3, was calculated (as the slope of the linear model deduced from the evolution of the pH as a function of time (ΔpH/Δtime) for value of pH between 6 and 5.3). Moreover, the pH_{STOP} corresponding to the pH value at V0 (corresponding to a velocity which definitively becomes non-detectable, i.e., below 0.1 mupH/minutes (0.0001 UpH/min)] was determined.

Acidification of milk by DGCC12455 and by DGCC7984 were found similar all along the kinetic. On the contrary, DGCC12456 displayed a distinct acidification profile (FIG. 1A). Indeed, upon about 600 min of fermentation with DGCC12456, the pH tended to stabilize around 4.37 and did not change until the end of the fermentation time (pH_{STOP}=4.37), whereas with the DGCC12455 and DGCC7984 strains, the pH kept decreasing after 600 min of fermentation and reached values around 4.1 and 4.2 at the end of the fermentation time. This peculiar acidification profile with a pH stabilization was named full-STOP phenotype. However, despite this peculiar kinetic at the end of the fermentation, the slope of acidification between 6 and 5.3 was 106 mUpH/min which is a speed of acidification that is expected in industrial dairy fermentation.

### Example 2: Identification of a Genetic Difference in the lacZ Gene of DGCC12456

Genomes of strains DGCC7984 and DGCC12456 were sequenced and compared. Among others, a difference between the two strains was identified in the lacZ gene. The lacZ gene is described (van den Bogaard et al., 2000; Vaughan et al., 2001) as encoding the β-galactosidase, an enzyme responsible for the hydrolysis of lactose into glucose and galactose. In DGCC12456 genome, a C base was replaced by a T base at position 1060 of the lacZ gene, leading to a non-conservative amino acid change, the substitution of an arginine by a cysteine, at position 354 (R354C substitution) of the β-galactosidase enzyme. Thus, the DGCC7984 has a lacZ allele encoding a β-galactosidase the sequence of which is as defined in SEQ ID NO:2, whereas the DGCC12456 strain has a lacZ allele encoding a β-galactosidase the sequence of which is as defined in SEQ ID NO:4. In contrast, sequencing of the lacZ gene of strain DGCC12455 revealed that its lacZ sequence was identical to that of DGCC7984 (i.e., encoding a β-galactosidase the sequence of which is as defined in SEQ ID NO:2). Altogether, these results suggested that the mutation in the lacZ gene may be responsible for the peculiar acidification profile of DGCC12456.

To further investigate this hypothesis, the β-galactosidase encoded by the lacZ gene of other S. thermophilus strains were compared. The R354C substitution found in DGCC12456 was not found in any of the β-galactosidase sequence of the other S. thermophilus strains, confirming that this substitution is unique to DGCC12456.

Most of the S. thermophilus strains that were tested bears a lacZ allele encoding a β-galactosidase the sequence of which is as defined in SEQ ID NO:2. In some S. thermophilus strains, amino acid differences compared to SEQ ID NO:2 have been identified. These identified amino acid differences were conservative substitutions and have led to the identification of 8 different β-galactosidase variant types (as defined herein), the sequence of which is as defined in SEQ ID NO: 6, 9, 12, 15, 18, 21, 24 and 27 [variants 1 to 8—Table 2].

TABLE 2
Comparative amino-acid sequence analysis of β-galactosidases encoded by S. thermophilus
strains. Numbering of amino-acid position is made accordingly to SEQ ID NO: 2.

| | Amino acid position (SEQ ID NO: 2 used for numbering) | | | | | | | | | | | % | SEQ |
|---|---|---|---|---|---|---|---|---|---|---|---|---|---|
| Type | 35 | 237 | 339 | 354* | 542 | 714 | 777 | 951 | 955 | 999 | 1002 | similarity | ID |
| DGCC7984 | E | A | V | R | Y | E | V | A | A | T | A | 100% | 2 |
| Variant 1 | A | T | V | R | Y | E | V | A | A | T | A | 99.7% | 6 |
| Variant 2 | E | A | V | R | Y | E | I | A | A | S | S | 99.7% | 9 |
| Variant 3 | E | A | V | R | Y | E | V | A | A | S | A | 99.9% | 12 |
| Variant 4 | E | A | V | R | Y | K | V | A | A | T | A | 99.9% | 15 |
| Variant 5 | E | A | V | R | F | E | V | A | A | T | A | 99.9% | 18 |
| Variant 6 | E | A | V | R | Y | E | V | S | A | T | A | 99.9% | 21 |
| Variant 7 | E | T | I | R | Y | E | V | A | A | T | A | 99.8% | 24 |
| Variant 8 | E | A | V | R | Y | E | V | A | V | T | A | 99.9% | 27 |
| DGCC12456 | E | A | V | C | Y | E | V | A | A | T | A | 99.9% | 4 |

*indicates the position 354 that differs in SEQ ID NO: 4

### Example 3: Comparison of the Acidification Profile of S. thermophilus Strain DGCC715 and DGCC11231, and their Derivatives Coding for a β-Galactosidase with the Sequence SEQ ID NO:4 Instead of SEQ ID NO:2 (R354C Substitution)

Derivatives of the strains DGCC715 and DGCC11231, named 715^{R354C} and 11231^{R354C} respectively, were constructed. The lacZ gene of DGCC12456 (encoding a β-galactosidase with a cysteine (C) at position 354) was inserted in lieu of the lacZ gene of the strains DGCC715 and DGCC11231. Practically, the lacZ gene was PCR amplified from DGCC12456 DNA. Competent cells of DGCC715 or DGCC11231 were prepared and transformed with the amplified DNA. Transformants were verified by sequencing.

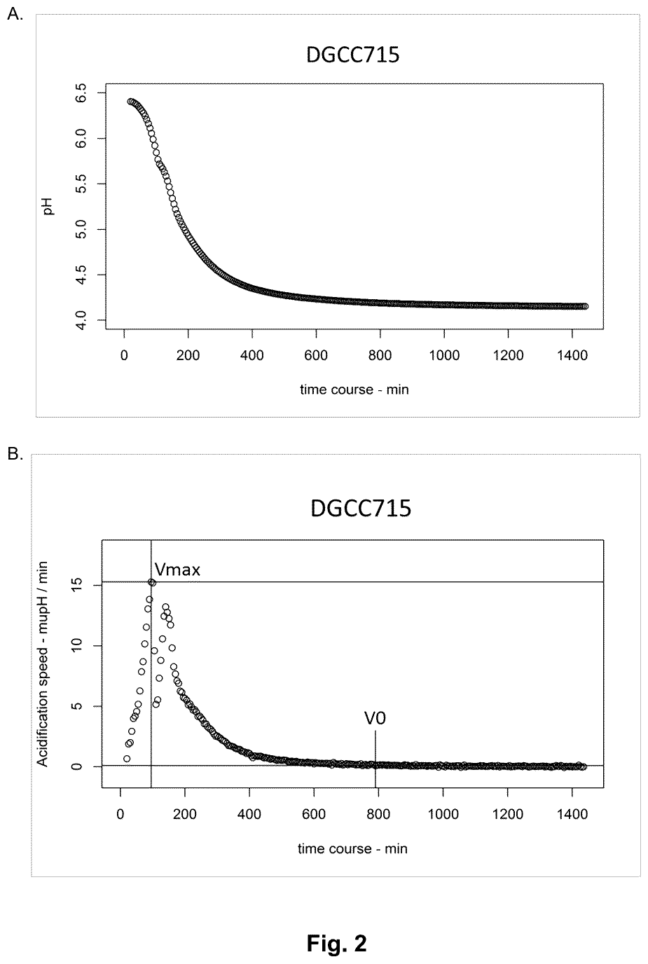

The ability of S. thermophilus strains DGCC715, DGCC11231, 715^{R354C} and 11231^{R354C} to ferment milk was evaluated as described in materiel and methods section [assay C]. The pH was recorded over time using a CINAC apparatus and the results are displayed in FIGS. 2A, 3A, 4A and 5A. The following descriptors were calculated (Table 3):
the slope between pH 6.0 and pH 5.3 (UpH/minute) [Slope pH6-5.3]; and
the pH_{STOP} corresponding to the pH value at V0 [corresponding to a velocity which definitively becomes non-detectable, i.e., below 0.1 mupH/minutes (0.0001 UpH/min)].

TABLE 3
Descriptors of the acidification kinetic of milk by DGCC715,
DGCC11231 and their constructed derivatives calculated from
the acidification curves

| | Slope pH 6-5.3 | |
|---|---|---|
| Strain | (10⁻⁴ UpH/min) | pH_{STOP} |
| DGCC715 | 109 | 4.19 |
| 715^{R354C} | 117 | 4.38 |
| DGCC11231 | 130 | 4.10 |
| 11231^{R354C} | 149 | 4.27 |

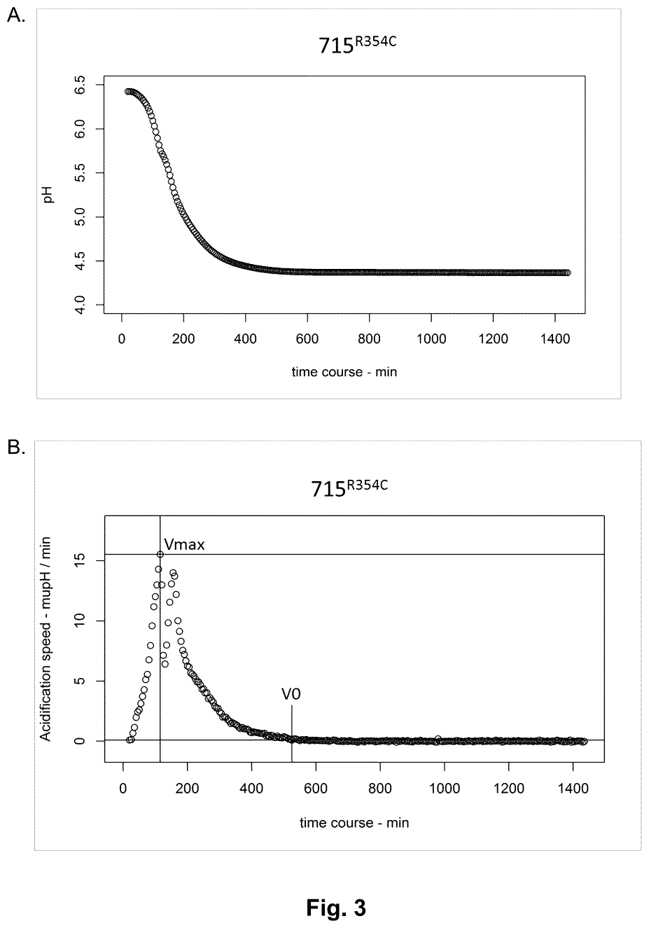

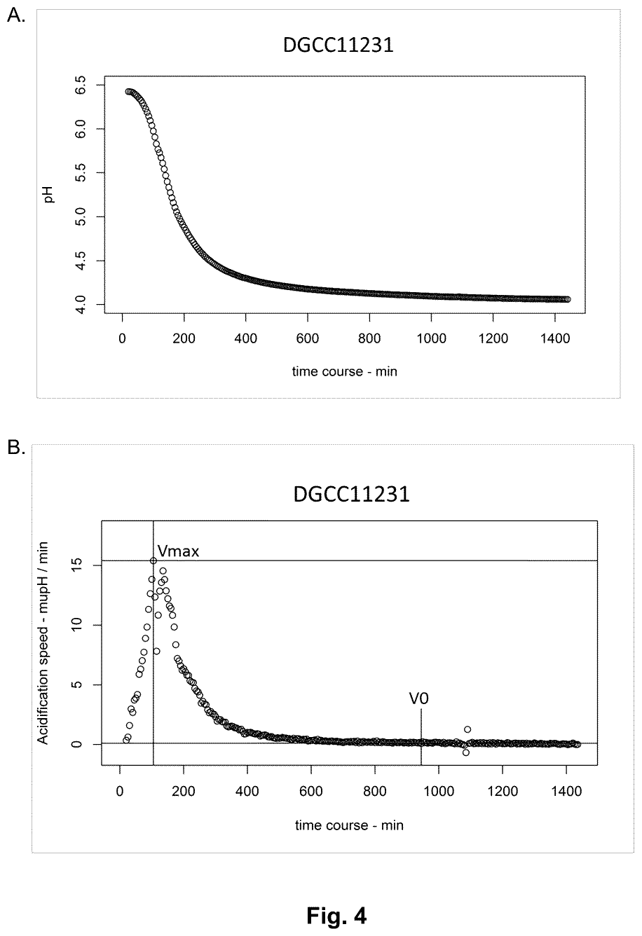

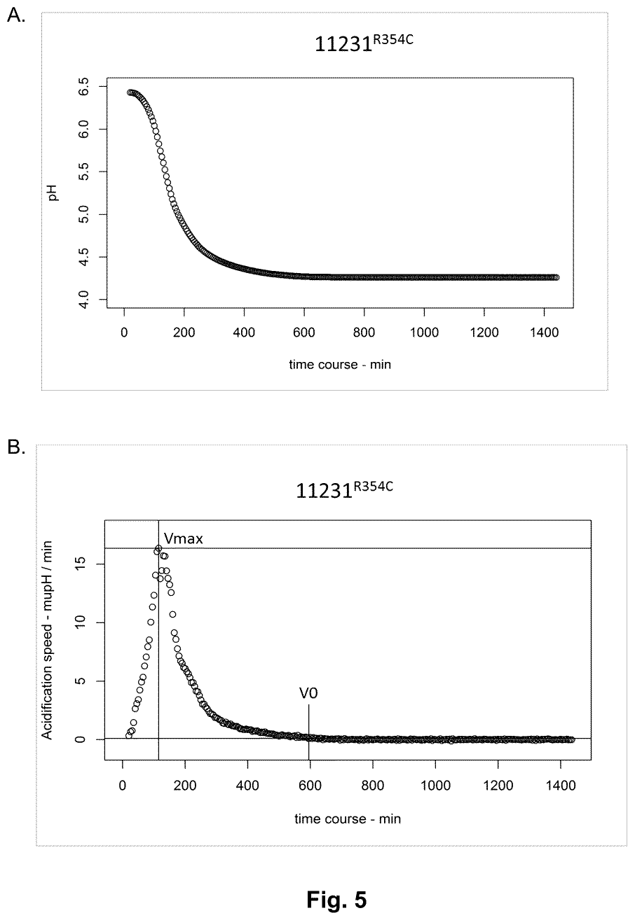

The results indicated that the acidification profile of the derivatives 715^{R354C} and 11231^{R354C} (see FIGS. 3A and 5A) differed from that of their respective parental strain (FIGS. 2A and 4A respectively) by a stabilization of the pH after 10 to 12 h of incubation. Stabilization of the pH (pH_{STOP}) occurred around pH 4.27 for 11231^{R354C} and pH 4.38 for 715^{R354C}, while the parental strains continued to acidify the milk after 12 hours of incubation to reach a pH of 4.19 and 4.10 respectively at the end of the incubation time. The results also indicated that, despite the substitution of an arginine by a cysteine in position 354 of the β-galactosidase, the slope of acidification between pH 6.0 and 5.3 was not negatively affected. As a consequence, the constructed derivatives were still appropriate to conduct dairy fermentation in industrial set-ups.

A second set of descriptors was also considered to characterize the full-STOP phenotype. This second set of descriptors was also determined for the DGCC12456 strain. For this purpose, the evolution of velocity (speed of acidification) as a function of time was calculated and the results are presented in FIGS. 1B, 2B, 3B, 4B and 5B. From these curves, the following descriptors were determined (Table 4):
the time to the maximal velocity obtained during the fermentation experiment (T_{Vmax}), time calculated (in minutes) as from the start of fermentation experiment;
the time to the pH_{STOP} (TpH_{STOP}) [the time to reach V0 as defined above], time calculated (in minutes) as from the start of fermentation experiment;
the time difference between TpH_{STOP} and T_{Vmax} (in minutes).

TABLE 4
Descriptors of the velocity kinetic of the fermentation by DGCC715,
DGCC11231 and their constructed derivatives and DGCC12456
calculated from the velocity curves

| | | | Δ time between T_{Vmax} |
|---|---|---|---|
| Strain | T_{Vmax} | TpH_{STOP} | and TpH_{STOP} |
| DGCC715 | 95 | 790 | 695 |
| 715^{R354C} | 115 | 525 | 410 |
| DGCC11231 | 105 | 945 | 840 |
| 11231^{R354C} | 115 | 595 | 480 |
| DGCC12456 | 160 | 610 | 450 |

The results showed that the time difference between TpH_{STOP} and T_{Vmax} was 410 and 480 minutes for the derivatives 715^{R354C} and 11231^{R354C} as compared to 695 and 840 minutes for their respective parental strains (Table 4). The results also showed that the DGCC12456 strain has the same profile as the derivatives 715^{R354C} and 11231^{R354C}. These results indicated that the time difference between TpH_{STOP} and T_{Vmax} of the derivatives 715^{R354C} and 11231^{R354C} was significantly decreased as compared to that of their respective parental strain (285 and 360 minute-difference respectively). These data reflected the ability of the derivatives 715^{R354C} and 11231^{R354C} when used to ferment milk, to achieve a stabilized pH (pH_{STOP}), which is higher, in a shorter time (as from the T_{Vmax}). These results confirmed that the R354C substitution in the β-galactosidase of DGCC12456 is responsible for the full-STOP phenotype.

Thus, the strains bearing a lacZ allele encoding a β-galactosidase with a cysteine at position 354 open the possibility of manufacturing fermented milks not only reaching their target pH (pH_{STOP}) in an acceptable industrial time (around 600 minutes), but also stabilizing their pH at fermentation temperature for up to 24 hours. In contrast, the parental strains continue to acidify milk until 700 to 800 minutes and at a lower pH, thus requiring stopping the fermentation process by a cooling step before the pH decreases too low.

### Example 4: Beta-Galactosidase Activities at pH6 and pH 4.5 for a Diversity of S. thermophilus Strains

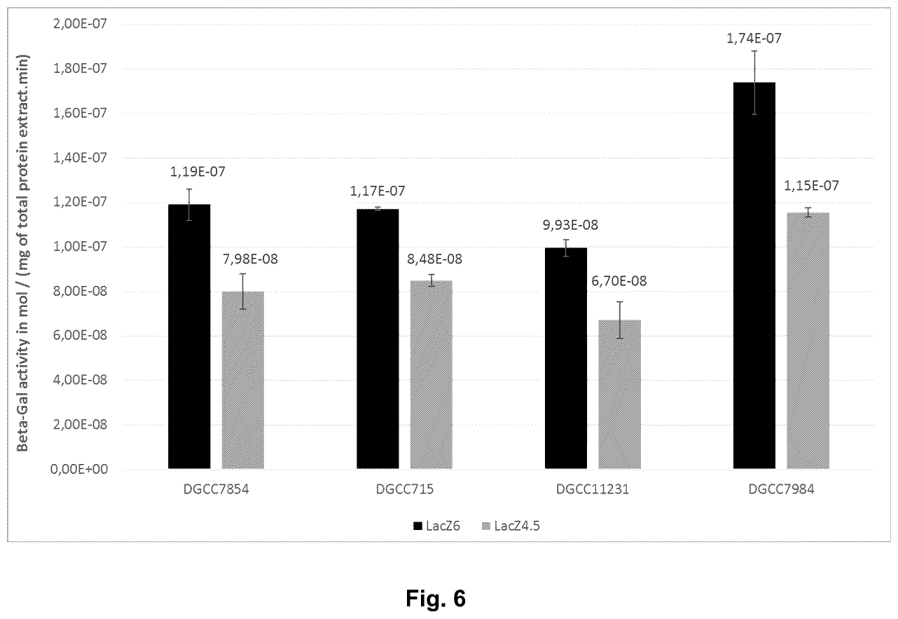

The β-galactosidase activities at pH 4.5 and pH 6 of a diversity of S. thermophilus strains bearing a lacZ allele encoding a β-galactosidase as defined in SEQ ID NO:2 was determined by assay B (as defined in the material and methods). The results are represented in FIG. 6.

First, these data showed that for a specific strain, its β-galactosidase activity at pH 4.5 is always less than its β-galactosidase activity at pH 6.0, traducing that the β-galactosidase activity decreases with the pH decrease.

Moreover, these data showed that there is an important variability in the β-galactosidase activity between strains bearing the same lacZ allele not only at pH 6.0 [from to 9.93×10⁻⁸ to 1.74×10⁻⁷ mol/(mg of total protein extract·min)] but also at pH 4.5 [from 6.7×10⁻⁸ to 1.15×10⁻⁷ mol/(mg of total protein extract·min)]. This variability can be explained by the genetic background specific to each strain. These data rose doubts on the fact that the β-galactosidase activity alone (at pH 4.5 and/or pH 6) can be used as a reliable descriptor to characterize the strains of the invention (having a full-STOP phenotype).

### Example 5: Comparison of Beta-Galactose Activity at pH6 and pH 4.5 of S. thermophilus Strain 715 and ST11231, their Derivatives 715^{R354C} and 11231^{R354C} and Strain DGCC12456

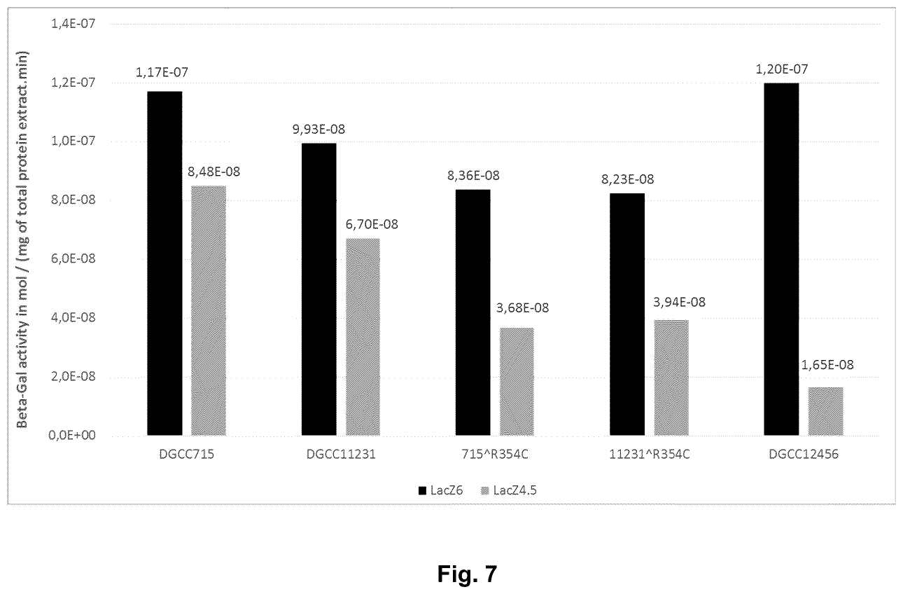

Upon the identification of the R354C substitution in the β-galactosidase and its role in the peculiar kinetic of acidification of milk by DGCC12456 (full-STOP phenotype), the β-galactosidase activity at pH 6 and at pH 4.5 of the strains DGCC715, DGCC11231, their respective constructed derivatives and DGCC12456, was determined by assay B (as defined in the material and methods). The results are represented in FIG. 7.

These data confirmed that the β-galactosidase activity at pH 4.5 of the strains bearing a lacZ allele encoding a β-galactosidase as defined in SEQ ID NO:4 (cysteine at position 354) is less than the β-galactosidase activity at pH 6.0.

It is noteworthy that the difference of β-galactosidase activity between pH 6 and pH4.5 is more important for the strains bearing a lacZ allele encoding a β-galactosidase as defined in SEQ ID NO:4 than for the strains bearing a lacZ allele encoding a β-galactosidase as defined in SEQ ID NO:2. Thus, the β-galactosidase activities at pH 4.5 of the strains bearing a lacZ allele encoding a β-galactosidase as defined in SEQ ID NO:4 was lower than the one of the strains bearing a lacZ allele encoding a β-galactosidase as defined in SEQ ID NO:2).

However, the variability in the β-galactosidase activity at pH 4.5 existing between strains bearing the same lacZ allele [from 1.65×10⁻⁸ to 3.94×10⁻⁸ mol/(mg of total protein extract·min) for strains bearing a lacZ allele encoding a β-galactosidase as defined in SEQ ID NO:4] confirmed that the β-galactosidase activity, even at pH4.5, cannot be used as the sole parameter to best characterize the strains of the invention having a full STOP phenotype

### Example 6: Investigation of Lactose Permease Activity (LacS)

In S. thermophilus, the lacZ gene is part of the lac operon (together with the lacS gene coding a lactose permease), and both the lactose permease and the β-galactosidase are involved in the catabolism of the lactose (by importing the lactose (LacS) and then hydrolysing it into glucose and galactose (lacZ).

The LacS activities at pH 6.0 and pH 4.5 of the strains DGCC715, DGCC11231, their respective derivatives and DGCC7984 and DGCC12456 strains, were determined by assay A (as defined in the material and methods). The results are represented in Table 5 (together with the β-galactosidase activity determined in example 4)

TABLE 5
LacS activity, LacZ activity and ratio at pH 4.5 and pH 6 of the DGCC715, DGCC11231,
their constructed derivatives, and DGCC7984 and DGCC12456 strains

| | LacS activity | | LacZ activity (mol/mg of | | Ratio LacS/ | |
|---|---|---|---|---|---|---|
| | (μmol/uDO · min) | | total protein extract · min) | | LacZ × 10⁻⁶ | |
| Strain | pH 6 | pH 4.5 | pH 6 | pH 4.5 | pH 6 | pH 4.5 |
| DGCC715 | 0.3696 | 0.1532 | 1.17 × 10⁻⁷ | 8.48 × 10⁻⁸ | 3.16 | 1.81 |
| 715^{R354C} | 0.2846 | 0.5036 | 8.36 × 10⁻⁸ | 3.68 × 10⁻⁸ | 3.40 | 13.70 |
| DGCC11231 | 0.7686 | 0.3347 | 9.93 × 10⁻⁸ | 6.70 × 10⁻⁸ | 7.74 | 5.00 |
| 11231^{R354C} | 0.5567 | 0.9075 | 8.23 × 10⁻⁸ | 3.94 × 10⁻⁸ | 6.77 | 23.05 |
| DGCC7984 | 0.4574 | 0.2943 | 2.10 × 10⁻⁷ | 1.11 × 10⁻⁷ | 2.18 | 2.66 |
| DGCC12456 | 0.4568 | 0.4529 | 1.20 × 10⁻⁷ | 1.65 × 10⁻⁸ | 3.82 | 27.49 |

While the lactose permease (LacS) activities at pH 4.5 were reduced compared to pH 6.0 for the strains coding for a β-galactosidase as defined in SEQ ID NO:2, these activities were increased (715^{R354C} and 11231^{R354C}) or unchanged (DGCC12456) for the strains coding for a β-galactosidase as defined in SEQ ID NO:4. It is hypothesized that to compensate a decrease in lactose hydrolysis by the β-galactosidase^{FS}, more lactose is imported by the lactose permease.

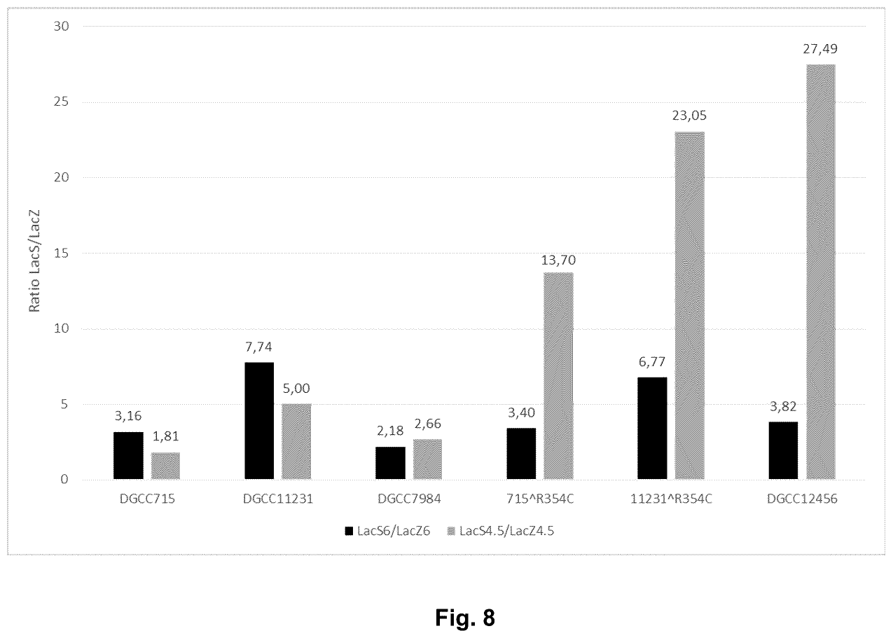

Therefore, the ratio LacS over LacZ (LacS/LacZ, which represents the efficiency for a strain to hydrolyse imported lactose=EH) at pH 4.5 and pH 6 was calculated (as defined herein) and is given in Table 5 and in FIG. 8. The strains bearing the lacZ allele encoding a β-galactosidase as defined in SEQ ID NO:2 displayed LacS/LacZ ratios of similar or slightly reduced values at pH 4.5 compared to pH 6.0. On the contrary, these ratios were significantly increased at pH 4.5 compared to pH 6.0 for the strains bearing a lacZ allele encoding a β-galactosidase as defined in SEQ ID NO:4. These results reflect a decrease of the efficiency of the strains of the invention in using the lactose of the medium (i.e., in hydrolysing the imported lactose) at pH 4.5 as compared to strains bearing the lacZ allele encoding a β-galactosidase as defined in SEQ ID NO:2.

The difference between the ratio LacS/LacZ at pH 4.5 of the strains bearing a lacZ allele encoding a β-galactosidase as defined in SEQ ID NO:2 and the ratio of the strains bearing a lacZ allele encoding a β-galactosidase as defined in SEQ ID NO:4 is highly significant, such that this parameter can be reliably used to characterize the strains of the invention.

The ratios LacS/LacZ at pH 4.5 of the strain DGCC715 and its derivative have been shown to be sufficiently discriminating, to use the DGCC715 strain in order to identify additional lacZ alleles encoding a β-galactosidase according to the invention (lacZ^{FS} alleles).

### Example 7: Efficiency of Hydrolysis of the Imported Lactose (EH) of S. thermophilus Strain 715 and ST11231, their Derivatives 715^{R354C} and 11231^{R354C} and Strain DGCC12456

Finally, the inventors have determined an additional descriptor representing the overall behavior of the S. thermophilus strain of the invention with respect to lactose metabolism during the whole process of milk fermentation. Thus, the following formula (I), representing the difference of efficiency of hydrolysis of imported lactose between pH 6.0 and pH 4.5 (EH_{pH6}−EH_{pH4.5}), was developed:

$$Δ EH = ln [ \frac{L a c {S}_{p H 6}}{L a c {Z}_{p H 6}} ] - ln [ \frac{{LacS}_{p H 4.5}}{L a c {Z}_{p H 4.5}} ] \qquad ( I )$$

In this formula, a ΔEH value around 0 or slightly positive or slightly negative means that the efficiency of hydrolysis of the imported lactose is similar at pH 6.0 and at pH 4.5 (i.e., that the efficiency of hydrolysis is not dependent upon the pH). In contrast, a significantly negative ΔEH value means that the efficiency of hydrolysis of the imported lactose is lower at pH 4.5 than at pH 6.0 (i.e., that the efficiency of hydrolysis significantly decreases with the pH decrease).

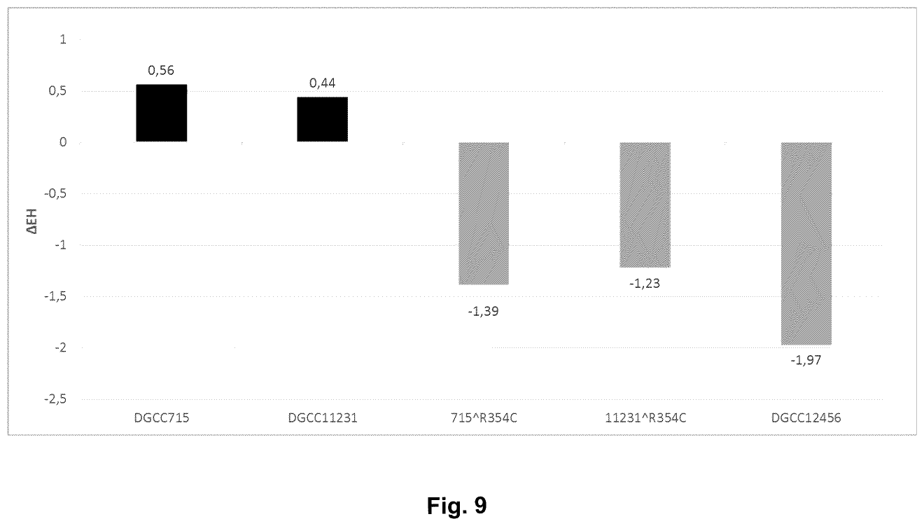

This formula was applied to calculate the ΔEH for the strains DGCC715, DGCC11231, their respective derivatives and DGCC12456, based on the β-galactosidase activity and lactose permease activities reported in Table 5. The results are presented in FIG. 9.

As show in FIG. 9, and as expected, the 2 S. thermophilus strains bearing a lacZ allele encoding a β-galactosidase as defined in SEQ ID NO:2 has a ΔEH value which is slightly positive (0.44 and 0.56). In contrast, the 3 S. thermophilus strains bearing a lacZ allele encoding a β-galactosidase as defined in SEQ ID NO:4 has a ΔEH value which is significantly negative (from −1.23 to −1.97).

In addition to the ratio LacS over LacZ at pH 4.5 defined above, the ΔEH value as defined by the formula (I) is a reliable parameter, enabling to characterize the strains of the invention having a full-STOP phenotype.

### Example 8: Impact of the Temperature of Packing During Manufacture of Stirred Yoghurt

A stirred yoghurt was prepared by inoculating a milk substrate (protein 3.9%, fat 1.5% and sucrose 6%) with the DGCC12456 strain described previously (at least 10⁷ cfu/ml) and a Lactobacillus bulgaricus (about 103 cfu/ml), and incubating the inoculated milk at 43° C. until pH=4.60 was reached. Right after, the yoghurt was stirred. Then, the stirred yoghurt was cooled and packed either at 20° C. or 35° C., and then stored at 10° C. along shelf-life (45 days).

The pH during shelf-life was measured using single probe portative pH-meter.

The viscosity at day 14 (after end of fermentation) was determined thanks to a Brookfield DV-I™ Prime viscometer (AMETEK Brookfield) using spindle S-05 and speed 10 rpm; after 30 seconds, the value of viscosity (in centipoise; cP) was determined.

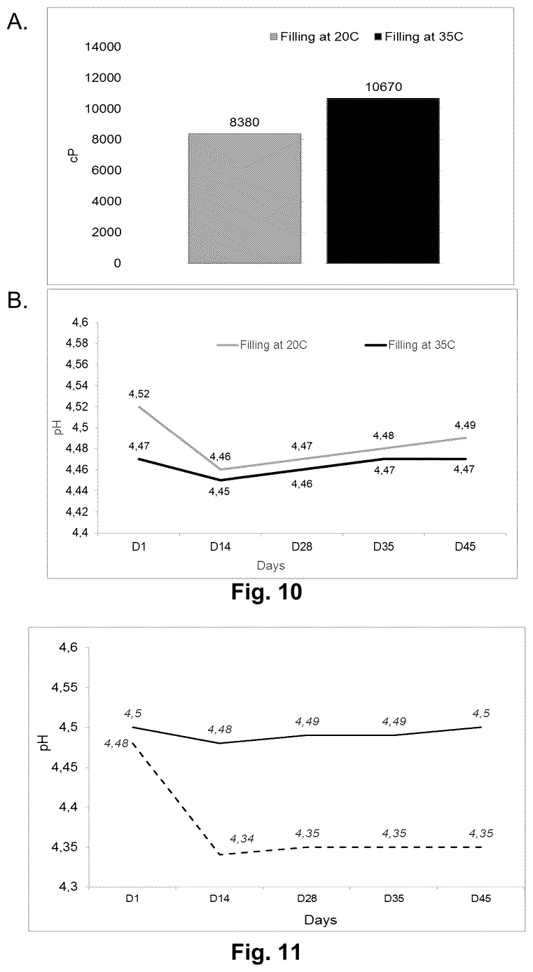

As shown in FIG. 10A and as expected, packing at 35° C. gave the stirred yoghurt a higher texture at day 14 as compared to packing at 20° C. (FIG. 10A). Interestingly, the pH of the stirred yoghurt was maintained at a high level for at least 45 days whatever the packing temperature (FIG. 10B).

These results confirm that a Streptococcus thermophilus strain of the invention having a full STOP phenotype presents a high interest for stirred yoghurt manufacturers, since enabling to improve the texture of the stirred yoghurt by increasing the temperature of packing while at the same time not compromising on the pH during storage.

### Example 9: Yoghurt Post-Acidification at 10° C.

A yoghurt was prepared by inoculating a milk substrate (protein 3.9% and fat 1.5%; no added sugar) with either (A) the DGCC12456 strain described previously (at least 10⁷ cfu/ml) and a Lactobacillus bulgaricus (about 103 cfu/ml) or (B) a reference starter culture with high post-acidification control performance consisting of Streptococcus thermophilus and Lactobacillus bulgaricus strains (the same L. bulgaricus strain as composition A) and by incubating the inoculated milk at 43° C. until pH=4.60 was reached. Right after, the yoghurt was cooled at 22° C. and then stored at 10° C. along shelf-life (45 days). The pH during shelf-life was measured using single probe portative pH-meter.

As shown in FIG. 11, both cultures showed a relatively high pH during the shelf-life. The reference starter culture showed a rapid pH decrease down to 4.34 up to day 14 and then a pH stability from day 14 to day 45 (dashed line); in contrast, the culture comprising the DGCC12456 strain showed a stable pH all over the shelf-life from day 1 to day 45 (pH between 4.48 and 4.5) (plain line).

These results confirm that a Streptococcus thermophilus strain of the invention having a full STOP phenotype presents a high interest for fermented milk manufacturers, since enabling to store fermented milks products at a temperature higher than the temperature of conventional cold room (typically less than 8° C.), without impacting the pH.

Altogether, the Streptococcus thermophilus strain of the invention offers fermented milk and yoghurt manufacturers new possibilities to improve their processes and reduce their costs, for example by making use of the pH stability at fermentation temperature for up to 24 hours in the manufacture of set yoghurt, by making use of both the texture improvement and pH stability when packing at high temperature in the manufacture of stirred yoghurt, or by making use of the pH stability at 10° C. for at least 45 days in the storage of their fermented milks.

1. A polynucleotide encoding a β-galactosidase^{FS}, which consist of an amino acid sequence identified by SEQ ID NO:4; and which is defined as a lacZ allele which increases the ratio of the activity of lactose importation of the LacS permease at pH 4.5 over the activity of lactose hydrolysis of the beta-galactosidase at pH 4.5 above 8 in a DGCC715 derivative, said DGCC715 derivative being a strain DGCC715 (deposited at the DSMZ on Feb. 12, 2019 under the accession number DSM33036), into which its lacZ gene was replaced by said polynucleotide encoding a β-galactosidase^{FS}.

2. A Streptococcus thermophilus strain comprising an allele of the lacZ gene as defined in SEQ ID NO:3, which is a lacZ^{FS} allele encoding a β-galactosidase^{FS} according to claim 1.

3. A bacterial composition comprising the Streptococcus thermophilus strain of claim 2, and one or more further lactic acid bacteria selected from the group consisting of Streptococcus, Lactococcus, Lactobacillus, Leuconostoc, Pediococcus, Enterococcus, Oenococcus and Bifidobacterium.

4. A food or feed product comprising the Streptococcus thermophilus strain of claim 2 or a bacterial composition comprising the Streptococcus thermophilus strain of claim 2, wherein the food or feed product is a dairy, meat or cereal food or feed product.

5. A method for manufacturing a fermented product, comprising:
a) inoculating a substrate with the Streptococcus thermophilus strain of claim 2 or a bacterial composition comprising the Streptococcus thermophilus strain of claim 2; and
b) fermenting the inoculated substrate obtained from step a) to obtain a fermented product.

6. A method according to claim 5, for manufacturing stirred yoghurt, comprising:
a) fermenting a milk substrate inoculated with the Streptococcus thermophilus strain of claim 2 or a bacterial composition comprising the Streptococcus thermophilus strain of claim 2, to obtain a stirred yoghurt with a pH from 4.2 to 4.7;
b) cooling the stirred yoghurt;
c) packing the stirred yoghurt; and
d) transferring the packages of step c) into a storage cold room;
wherein the temperature of cooling and packing is at least 24° C.

7. A method according to claim 5, for manufacturing stirred yoghurt, comprising:
a) fermenting a milk substrate inoculated with the Streptococcus thermophilus strain of claim 2 or a bacterial composition comprising the Streptococcus thermophilus strain of claim 2, to obtain a stirred yoghurt with a pH from 4.2 to 4.7;
b) packing the stirred yoghurt; and
c) transferring the packages of step b) into a storage cold room;
wherein the process does not comprise any cooling step between end of fermentation and packing.

8. A method according to claim 5, for manufacturing set yoghurt, comprising:
a) packing a milk substrate inoculated with the Streptococcus thermophilus strain of claim 2 or a bacterial composition comprising the Streptococcus thermophilus strain of claim 2;
b) fermenting the inoculated milk substrate to obtain a set yoghurt with a pH from 4.2 to 4.7; and
c) directly transferring the packages of step b) into a storage cold room, wherein said process does not comprise a cooling step in a cooling room after the fermentation step b).

9. A method to prepare a Streptococcus thermophilus strain with a full STOP phenotype, comprising:
a) providing a Streptococcus thermophilus strain, having a ratio of the activity of lactose importation of the LacS permease at pH 4.5 over the activity of lactose hydrolysis of the beta-galactosidase at pH 4.5 which is less than 5;
b) replacing the allele of the lacZ gene of said Streptococcus thermophilus strain of step a) with a polynucleotide according to claim 1, or replacing a part of the allele of the lacZ gene of said Streptococcus thermophilus strain of step a) with a polynucleotide comprising a part of at least 100 nucleotides of the sequence of said β-galactosidase^{FS} comprising or consisting of an amino acid sequence identified by SEQ ID NO:4, wherein said nucleotide part encompasses the codon corresponding to the residue 354 of said β-galactosidase^{FS}, or modifying the sequence of the lacZ gene of said Streptococcus thermophilus strain of step a) to have a lacZ^{FS} allele with the same sequence as a polynucleotide according to claim 1; and
c) recovering the Streptococcus thermophilus strain(s) with a full STOP phenotype when used to ferment milk.


SEQUENCE LISTING (38 sequences)

SEQ ID NO: 1 - DNA, 3081 bp, Streptococcus thermophilus
  source 1..3081: mol_type = other DNA
atgaacatgactgaaaaaattcaaacttatttaaacgatccaaagattgttagcgttaatactgttgatgctcactcagatcataagtattttgaatctcttgaagaattttctgaaggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccaatatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttactctacagcaagctggttggaagaccaagacttctggagactttacggtatttttagagatgtttacttgtatgctattccaaaagttcacgttcaagatctctttgttaagggagattatgattaccaaacaaaagcaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggattcagacgctttgaaattaaagataaacttatgcttttgaatggtaagagaattgtctttaaaggggttaacagacacgaatttaacgctagaacaggacgttgtatcactgaagaagatatgctttgggatatcaaagtgatgaagcaacataacatcaatgctgttcgtacttcacactatcctaaccaaacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagtcgaggcttcaaattctgaacaaatttacgaagttgcttgtgttctgagggaagcaactgaatgggctcctaaaggtcatgaaattgttcgtggtcaatatgttgttgaaaagattagcactgaaacaccagttaaagcacctttgaatgttgttgaaggcgacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgattacaagggtcgaatctttgttactgcaaactatgatggtaaagcaggtttgccaaacttccctgaatttggtctagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacagtactgaacaaattgaaaatgctcgtcaccaatatgagttgcaagaatctgatgctacatggattaaagttcttgctgctcaaatgggtgtaggtggtgacgacacatggggtgctccagttcatgacgaattcttgcttagctcagcagatagctatcaattaagcttcatgattgaaccactaaattag

SEQ ID NO: 2 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 3 - DNA, 3081 bp, Streptococcus thermophilus
  source 1..3081: mol_type = other DNA
atgaacatgactgaaaaaattcaaacttatttaaacgatccaaagattgttagcgttaatactgttgatgctcactcagatcataagtattttgaatctcttgaagaattttctgaaggggagatgaagttaagacaatctcttaatggaaaatggaaaattcactatgctcagaatacaaatcaggttttaaaagacttttataaaacagaatttgatgaaactgatttgaatttcatcaatgtaccaggtcatttagagcttcaaggttttggttctccacaatatgtgaatacccaatatccttgggatggtaaagaattccttcgtccacctcaagttcctcaagaatcaaatgctgttgcatcatacgttaaacattttaccttgaatgatgcattaaaagataaaaaagtatttatctcattccaaggggttgctacttccatctttgtatgggtcaatggtaacttcgtaggatacagtgaagattcatttacacctagtgaatttgaaattagtgattaccttgttgaaggtgataacaagttggcggtagctgtttatcgttattctacagcaagctggttggaagaccaagacttctggagactttacggtatttttagagatgtttacttgtatgctattccaaaagttcacgttcaagatctctttgttaagggagattatgattaccaaacaaaagcaggtcaattggatattgatttgaagactgttggtgattatgaagacaagaagattaaatatgttctttcagattatgaaggcatcgttacagaaggtgatgcatctgttaatggtgacggtgaactatctgtaagtcttgaaaatcttaaaatcaaaccttggagtgctgaaagtcctaaactttacgatttgatccttcatgttttggatgatgaccaagttgttgaagtcgttccagttaaagttggatttagacgctttgaaattaaagataaacttatgcttttgaatggtaagagaattgtctttaaaggggttaacagacacgaatttaacgctagaacaggatgttgtatcactgaagaagatatgctttgggatatcaaagtgatgaaacaacataacatcaatgctgttcgtacttcacactatcctaaccaaacacgttggtatgaattgtgtgatgaatatggactttatgttatcgatgaagccaaccttgaaacacacggtacatggcaaaaacttggtctatgcgaaccttcatggaatatcccagctagtgaaccagaatggttgcctgcttgtttggatcgtgccaataacatgttccaacgcgataagaaccacgctagtgttatcatttggtcttgtggtaatgaatcatatgctggtaaagatattgctgacatggctgattacttccgtagtgttgacaatactcgtccagttcactatgaaggtgttgcatggtgtcgtgagtttgattacattacagacatcgaaagtcgtatgtatgcgaaaccagctgatatcgaagaatacctcacaactggtaaactagttgatctttcaagcgttagtgataaacactttgcttcaggtaacctaactaacaaacctcaaaaaccttatatttcatgtgaatacatgcacacaatgggtaactctggtggtggattgcaactctacactgacttagagaaatatccagaataccaaggtggatttatttgggacttcattgaccaagctatttacaaaacacttccaaatggtagcgaattcctatcatatggtggtgactggcatgatagaccttctgactacgaattttgtggaaatggtatcgtctttgcagatcgtaccctaactccaaaacttcaaacagttaaacatctttactctaatattaagattgctgttgatgaaaaatcagtaactatcaagaatgataatctcttcgaagatctttctgcttatactttcctagctagagtttacgaagatggtagaaaagttagtgaaagtgaatatcactttgatgttaaaccaggcgaagaagcaacattcccagttaactttgtagtcgaggcttcaaattctgaacaaatttacgaagttgcttgtgttctgagggaagcaactgaatgggctcctaaaggtcatgaaattgttcgtggtcaatatgttgttgaaaagattagcactgaaacaccagttaaagcacctttgaatgttgttgaaggcgacttcaacatcggtattcaaggacaaaacttctcaatcttgctttcacgtgcacaaaatactttagtatctgctaagtataatggtgttgaattcattgagaaaggtcctaaacttagcttcactcgtgcttacactgacaacgatcgtggtgctggatatccattcgaaatggcaggctggaaggttgctggaaactatagtaaagttacagatactcaaattcaaatcgaagacgactctgttaaagtgacttatgttcatgaattgccaggcttgtctgatgtcgaagttaaggtaacttatcaagttgattacaagggtcgaatctttgttactgcaaactatgatggtaaagcaggtttgccaaacttccctgaatttggtctagaatttgctatcggttcacaatttacaaaccttagctattatggatacggtgcagaagaaagctaccgtgataaacttcctggtgcctatcttggtcgatatgaaacatctgttgaaaagacatttgctccatatctaatgccacaagaatctggtaatcactatggtactcgtgaattcacagtatctgatgataaccataatggtcttaaattcaccgcacttaataaagcattcgaattcagtgctttgcgtaacagtactgaacaaattgaaaatgctcgtcaccaatatgagttgcaagaatctgatgctacatggattaaagttcttgctgctcaaatgggtgtaggtggtgacgacacatggggtgctccagttcatgacgaattcttgcttagctcagcagatagctatcaattaagcttcatgattgaaccactaaattag

SEQ ID NO: 4 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 5 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 6 - protein, 1026 aa, Streptococcus thermophilus
MNMTEKIQTYLNDPKIVSVNTVDAHSDHKYFESLAEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIIFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 7 - protein, 1026 aa, Streptococcus thermophilus
  VARIANT 354: note = "Xaa" is any amino acid except Arg
MNMTEKIQTYLNDPKIVSVNTVDAHSDHKYFESLAEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIIFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 8 - protein, 1026 aa, Streptococcus thermophilus
MNMTEKIQTYLNDPKIVSVNTVDAHSDHKYFESLAEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIIFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 9 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGIEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGSPVHDEFLLSSADSYQLSFMIEPLN

SEQ ID NO: 10 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGIEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGSPVHDEFLLSSADSYQLSFMIEPLN

SEQ ID NO: 11 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGIEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGSPVHDEFLLSSADSYQLSFMIEPLN

SEQ ID NO: 12 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGAPVHDEFLLSSADSYQLSFMIEPLN

SEQ ID NO: 13 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGAPVHDEFLLSSADSYQLSFMIEPLN

SEQ ID NO: 14 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGAPVHDEFLLSSADSYQLSFMIEPLN

SEQ ID NO: 15 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATKWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 16 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATKWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 17 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATKWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 18 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPFISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 19 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPFISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 20 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPFISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 21 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTSLNKAFEFSALRNSTEQIENARHQYELQESDATWIKVLAAQMGVGGDDTWGAPVHDEFLLSSADSYQLSFMIEPLN

SEQ ID NO: 22 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTSLNKAFEFSALRNSTEQIENARHQYELQESDATWIKVLAAQMGVGGDDTWGAPVHDEFLLSSADSYQLSFMIEPLN

SEQ ID NO: 23 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTSLNKAFEFSALRNSTEQIENARHQYELQESDATWIKVLAAQMGVGGDDTWGAPVHDEFLLSSADSYQLSFMIEPLN

SEQ ID NO: 24 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIIFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 25 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIIFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 26 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKTGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIIFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDTWGAPVHDEFLLSSADSYQLSFMIEPLN

SEQ ID NO: 27 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKVFEFSALRNSTEQIENARHQYELQESDATWIKVLAAQMGVGGDDTWGAPVHDEFLLSSADSYQLSFMIEPLN

SEQ ID NO: 28 - protein, 1026 aa, Streptococcus thermophilus
  VARIANT 354: note = "Xaa" is any amino acid except Arg
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGXCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKVFEFSALRNSTEQIENARHQYELQESDATWIKVLAAQMGVGGDDTWGAPVHDEFLLSSADSYQLSFMIEPLN

SEQ ID NO: 29 - protein, 1026 aa, Streptococcus thermophilus
MNMTEKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGCCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATEWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKVFEFSALRNSTEQIENARHQYELQESDATWIKVLAAQMGVGGDDTWGAPVHDEFLLSSADSYQLSFMIEPLN

SEQ ID NO: 30 - DNA, 1905 bp, Streptococcus thermophilus
  source 1..1905: mol_type = other DNA
atggaaaaatctaaaggtcagatgaagtctcgtttatcctacgcagctggtgcttttggtaacgacgtcttctatgcaaccttgtcaacatactttatcatgtttgtgacaactcacttgtttaacacaggtgatccaaagcaaaatagtcactacgtactattaatcactaacattatctctattttgcgtatcttggaagtatttatcgatccattgatcggtaatatgattgataacactaatactaagtatggtaaattcaaaccatgggtagttggtggtggtatcatcagttctatcaccttgttgcttctcttcaccgatttaggtggtttgaataaaacaaatcctttcttgtaccttgtactttttggaattatctaccttataatggatgtcttctactcgattaaagatatcggtttctggtcaatgcttcctgccttgtctcttgacagtcacgaacgtgaaaaaatggcaacttttgcccgtattggttctacgattggtgccaatattgtaggtgttgccatcatgccaatcgttttgttcttctctatgacgaacagtagtggctctggagataaatctggatggttctggtttgcatttatcgttgcactcattggtgtgattacatcaattgctgttggtattggtacacgtgaagttgagtcaaaacttcgtgataacaatgaaaaaactagccttaaacaagtctttaaagttcttggtaaaaacgaccaattgatgtggctgtctctcggatattggttctatggtcttggtattaatacacttaatgctcttcaactttattacttcacatttatccttggtgattcagggaaatactcaatcctttacggattgaatacagttgttggtttggtttcagtttcactgttccctagtcttgctggtaaattcaaccgtaaacgtttgttctatggatgtattgcagtaatgctcggtggtatcggaatctttagtattgcaggtacttcacttccaatgatcttgactgcagctgaactcttcttcattccacaacctcttgtgttccttgttatccttatgattatctctgactcagtagaatatggtcaatggaaattgggacaccgtgatgaatcacttactttgtcagttcgtccacttgttgataaacttggtggtgcgatgtcaaactggcttgtttctacaattgccgtagctgccggtatgacaacaggtgcctcagcatcaacaattacaacacatcaacagtctatttttaagcttagcatgtttggtttcccagcagcagcaatgcttatcggtgccttcattattgctcgtaaaatcactttgactgaagcacgtcacgctaaaattgttgaagaattggaacatcgctttagcgtagcaacttctgaaaatgaagttaaagctaacgtcgtatctcttgtaacccctacaactggttatttggttgatctctcaagtgttaatgatgaacactttgcttcaggtagcatgggtaaaggtttcgccattaaacctactgatggagctgtctttgcaccaattagtggtaccattcgtcaaattcttcctactcgccatgcagttggtattgaaagtgaagatggtgtcattgttcttatccacgttggcatcggaacagttaaacttaatggtgaaggattcattagttacgtagaacaaggtgatcgtgttgaagttggacaaaaacttcttgagttctggtcaccaattattgagaaaaatggtcttgatgacacagtacttgtcactgtaactaattcagaaaaattcagtgctttccatcttgaacaaaaagttggagaaaaggtagaagctttgtctgaagttattaccttcaaaaaaggagaataa

SEQ ID NO: 31 - protein, 634 aa, Streptococcus thermophilus
MEKSKGQMKSRLSYAAGAFGNDVFYATLSTYFIMFVTTHLFNTGDPKQNSHYVLLITNIISILRILEVFIDPLIGNMIDNTNTKYGKFKPWVVGGGIISSITLLLLFTDLGGLNKTNPFLYLVLFGIIYLIMDVFYSIKDIGFWSMLPALSLDSHEREKMATFARIGSTIGANIVGVAIMPIVLFFSMTNSSGSGDKSGWFWFAFIVALIGVITSIAVGIGTREVESKLRDNNEKTSLKQVFKVLGKNDQLMWLSLGYWFYGLGINTLNALQLYYFTFILGDSGKYSILYGLNTVVGLVSVSLFPSLAGKFNRKRLFYGCIAVMLGGIGIFSIAGTSLPMILTAAELFFIPQPLVFLVILMIISDSVEYGQWKLGHRDESLTLSVRPLVDKLGGAMSNWLVSTIAVAAGMTTGASASTITTHQQSIFKLSMFGFPAAAMLIGAFIIARKITLTEARHAKIVEELEHRFSVATSENEVKANVVSLVTPTTGYLVDLSSVNDEHFASGSMGKGFAIKPTDGAVFAPISGTIRQILPTRHAVGIESEDGVIVLIHVGIGTVKLNGEGFISYVEQGDRVEVGQKLLEFWSPIIEKNGLDDTVLVTVTNSEKFSAFHLEQKVGEKVEALSEVITFKKGE

SEQ ID NO: 32 - DNA, 19 bp, synthetic construct
  misc_feature 1..19: note = Oligonucleotide primer
  source 1..19: mol_type = other DNA
cttgactgcagctgaactc

SEQ ID NO: 33 - DNA, 21 bp, synthetic construct
  misc_feature 1..21: note = Oligonucleotide primer
  source 1..21: mol_type = other DNA
ctcgactacaaagttaactgg

SEQ ID NO: 34 - DNA, 20 bp, synthetic construct
  misc_feature 1..20: note = Oligonucleotide primer
  source 1..20: mol_type = other DNA
cagagttacccattgtgtgc

SEQ ID NO: 35 - DNA, 20 bp, synthetic construct
  misc_feature 1..20: note = Oligonucleotide primer
  source 1..20: mol_type = other DNA
aggttggcttcatcgataac

SEQ ID NO: 36 - DNA, 21 bp, synthetic construct
  misc_feature 1..21: note = Oligonucleotide primer
  source 1..21: mol_type = other DNA
catcaccttctgtaacgatgc

SEQ ID NO: 37 - DNA, 21 bp, synthetic construct
  misc_feature 1..21: note = Oligonucleotide primer
  source 1..21: mol_type = other DNA
gtaacttcgtaggatacagtg

SEQ ID NO: 38 - DNA, 20 bp, synthetic construct
  misc_feature 1..20: note = Oligonucleotide primer
  source 1..20: mol_type = other DNA
aggacgttgtatcactgaag